(19) United States
(12) Patent Application Publication
(10) Pub. No.: US 2025/0090218 A1
(43) Pub. Date: Mar. 20, 2025

(54) DEVICES FOR VALVE SPLITTING

(71) Applicant: RAMBAM MEDTECH LTD., Haifa (IL)

(72) Inventors: Arthur KERNER, Haifa (IL); Yair FELD, Haifa (IL); Eyal TEICHMAN, Hod Hasharon (IL); Shlomo LEWKOWICZ, Tivon (IL); Or ALON, Ramot Menashe (IL)

(73) Assignee: RAMBAM MEDTECH LTD., Haifa (IL)

(21) Appl. No.: 18/729,289

(22) PCT Filed: Jan. 18, 2023
(86) PCT No.: PCT/IL2023/050049

§ 371 Date: Jul. 16, 2024

Related U.S. Application Data
(60) Provisional application No. 63/300,298, filed on Jan. 18, 2022.

(51) Int. Cl. A61B 18/14; A61B 18/00

(52) U.S. Cl. CPC A61B 18/1445; A61B 2018/00369; A61B 2018/00601

(57) ABSTRACT

An electrosurgical instrument (20, 120, 220, 420, 620, 720) is provided for excising a segment of a heart valve leaflet, the electrosurgical instrument including a delivery shaft (26, 426) supporting a first jaw (24A, 124A, 224A, 424A, 624A) and a second jaw (24B, 124B, 224B, 424B, 624B), which together are configured to assume (i) an open position, in which the first and the second jaws are spaced apart to allow insertion of a portion of the leaflet therebetween, and (ii) a closed position, in which the first and the second jaws sandwich the inserted leaflet portion. The electrosurgical instrument (20, 120, 220, 420, 620, 720) also includes first and second electrically insulated wire portions (34A, 734A, 34B, 734B) and, longitudinally therebetween, an electrically exposed cutting portion (36, 736). When an electrical current is applied to the electrically exposed cutting portion (36, 736), movement of the electrically exposed cutting portion (36, 736) along a predefined path (42) excises the leaflet segment from the inserted leaflet portion, such that the excised leaflet segment remains entrapped between the first and the second jaws. Other embodiments are also described.

### CROSS-REFERENCE TO RELATED APPLICATIONS

The present application claims priority from U.S. Provisional Application 63/300,298, filed Jan. 18, 2022, which is incorporated herein by reference and assigned to the assignee of the present application.

### FIELD OF THE APPLICATION

The present invention relates generally to transcatheter interventional cardiology devices and methods, and specifically to transcatheter interventional cardiology devices and methods for cutting native or prosthetic heart valve leaflets.

### BACKGROUND OF THE APPLICATION

Transcatheter Aortic Valve Implantation (TAVI) has become common practice for aortic valve replacement for severe aortic stenosis (AS) and valve-in-valve procedures for biological prosthetic valve degeneration (see, for example, “Five-Year Outcomes of Transcatheter or Surgical Aortic-Valve Replacement,” Makkar R R, et al., N Engl J Med. 2020).

A rare catastrophic complication of TAVI is coronary artery obstruction, specifically in the case of prosthetic valves (see, for example, Tang G H L et al., “Assessing the Risk for Coronary Obstruction After Transcatheter Aortic Valve Implantation and the Need to Perform BASILICA: The VIVID Classification,” EuroIntervention. 2020 May 4) and TAVI-in-TAVI (see, for example, GHL et al., “Feasibility of Repeat TAVR After SAPIEN 3 TAVR A Novel Classification Scheme and Pilot Angiographic Study,” JACC: Cardiovascular Interventions Volume 12, Issue 13, July 2019).

US Patent Application Publication 2020/0146690 to Rothstein et al. describes methods, systems and devices for severing and optionally removing at least a portion of heart valve leaflets. Leaflets can be partially removed or entirely removed or otherwise, the leaflets can be severed or splayed in such a way as to avoid coronary blockage, LVOT obstruction, or access challenges in procedures where a prosthetic valve is to be implanted within a previously implanted prosthetic valve. Aspects of the disclosure also relate to numerous devices for and methods of disabling one or more valve ligating devices to provide an unobstructed valve opening so that a prosthetic heart valve can be implanted within the opening. The ligation device(s) is disabled either by removing the ligation device(s) or severing one leaflet so that ligated leaflets can be separated. In some embodiments, the ligation device(s) are severed to disable the ligation device(s).

PCT Publication WO 2022/013857 to Feld et al. describes a valve-splitting device for splitting a native or prosthetic valve cusp. The valve-splitting device includes a cover sheath and a splitter, which is removably disposed inside the cover sheath. The splitter includes a first over-the-wire cutter, which is shaped so as to define a guidewire lumen that is configured to accept and slide over a guidewire; and a second opposing cutter. The splitter is configured, upon distal advancement of the splitter out of a distal end of the cover sheath, to be opened such that the first over-the-wire cutter and the second opposing cutter are separated from each other and are disposable on opposite sides of the cusp, such that closing the splitter by bringing the first over-the-wire cutter and the second opposing cutter together cuts the cusp. Other embodiments are also described.

### SUMMARY OF THE APPLICATION

Some embodiments of the present invention provide an electrosurgical instrument for excising a segment of a native or prosthetic heart valve leaflet, typically before performing implantation of an artificial cardiac valve, in order to prevent coronary artery obstruction.

The electrosurgical instrument comprises a delivery shaft, which is shaped so as to define one or more longitudinal channels therethrough; first and second jaws, which are supported at a distal end portion of the delivery shaft; and a wire, which comprises first and second electrically insulated wire portions and, longitudinally therebetween, an electrically exposed cutting portion.

The first and the second jaws are shaped so as to define respective first and second leaflet-facing surfaces. The first and the second jaws together are configured to assume:
an open position, in which the first and the second jaws are spaced apart to allow insertion, between the first and the second jaws, of a portion of the leaflet including the leaflet segment to be excised; and
a closed position, in which the first and the second jaws sandwich the inserted leaflet portion.

At least one (optionally both) of the first and the second electrically insulated wire portions passes through the one or more longitudinal channels of the delivery shaft and is configured to couple the electrically exposed cutting portion in electrical communication with an electrosurgical generator unit.

The electrosurgical instrument is configured such that when the first and the second jaws are in the closed position sandwiching the inserted leaflet portion and an electrical current is applied to the electrically exposed cutting portion by the electrosurgical generator unit, movement of the electrically exposed cutting portion along a predefined path excises the leaflet segment from the inserted leaflet portion, such that the excised leaflet segment remains entrapped between the first and the second jaws.

Typically, the electrosurgical instrument is configured such that the predefined path is not entirely straight. Typically, the electrosurgical instrument is configured such that the predefined path includes one or more curved sections, such as exactly one curved section, and, optionally one or more straight sections. For example, the predefined path may be U-shaped.

For some applications, the first and the second leaflet-facing surfaces are shaped so as to define respective first and second grooves, which together define the predefined path when the first and the second jaws are in the closed position. At least when the first and the second jaws are in the closed position, the first and the second electrically insulated wire portions are slidably disposed partially within the first and the second grooves, respectively, with the electrically exposed cutting portion extending between respective planes defined by the first and the second grooves.

The electrosurgical instrument is configured such that when the first and the second jaws are in the closed position sandwiching the inserted leaflet portion and the electrical current is applied to the electrically exposed cutting portion by the electrosurgical generator unit, motion of the first and the second electrically insulated wire portions in the one or more longitudinal channels causes the movement of the electrically exposed cutting portion along the predefined path.

Thus, the first and second electrically insulated wire portions serve two functions: conducting the current to the electrically exposed cutting portion; and remotely conveying a mechanical force to the electrically exposed cutting portion to move the cutting portion.

In some applications, at least one of the first and second jaws comprises one or more teeth that are configured to pierce the leaflet segment to be excised, when the first and the second jaws are in the closed position, so as to fixate the excised leaflet segment between the first and the second jaws. This fixation ensures that the entire excised leaflet segment is removed from the vasculature with the jaws when they are withdrawn from the body.

The following Inventive Concepts are therefore provided in accordance with respective embodiments of the present invention:
1. An electrosurgical instrument for use with an electrosurgical generator unit, for excising a segment of a native or prosthetic heart valve leaflet, the electrosurgical instrument including:
   a delivery shaft, which is shaped so as to define one or more longitudinal channels therethrough;
   first and second jaws, which (a) are supported at a distal end portion of the delivery shaft, (b) are shaped so as to define respective first and second leaflet-facing surfaces, and (c) together are configured to assume (i) an open position, in which the first and the second jaws are spaced apart to allow insertion, between the first and the second jaws, of a portion of the leaflet including the leaflet segment to be excised, and (ii) a closed position, in which the first and the second jaws sandwich the inserted leaflet portion; and
   first and second electrically insulated wire portions and, longitudinally therebetween, an electrically exposed cutting portion, wherein at least one of the first and the second electrically insulated wire portions passes through the one or more longitudinal channels of the delivery shaft and is configured to couple the electrically exposed cutting portion in electrical communication with the electrosurgical generator unit,
   wherein the electrosurgical instrument is configured such that when the first and the second jaws are in the closed position sandwiching the inserted leaflet portion and an electrical current is applied to the electrically exposed cutting portion by the electrosurgical generator unit, movement of the electrically exposed cutting portion along a predefined path excises the leaflet segment from the inserted leaflet portion, such that the excised leaflet segment remains entrapped between the first and the second jaws.
2. The electrosurgical instrument according to Inventive Concept 1, wherein the electrosurgical instrument includes a wire that includes the first and the second electrically insulated wire portions and, longitudinally therebetween, the electrically exposed cutting portion.
3. The electrosurgical instrument according to any one of Inventive Concepts 1 and 2, wherein the native or prosthetic valve is a native or prosthetic aortic valve, and the electrosurgical instrument is configured to excise the segment of the native or prosthetic aortic valve.
4. The electrosurgical instrument according to any one of Inventive Concepts 1 and 2, wherein the native or prosthetic valve is a native mitral valve, and the electrosurgical instrument is configured to excise the segment of the native mitral valve.
5. The electrosurgical instrument according to any one of Inventive Concepts 1 and 2, wherein both the first and the second electrically insulated wire portions pass through the one or more longitudinal channels of the delivery shaft.
6. The electrosurgical instrument according to any one of Inventive Concepts 1 and 2, wherein the electrosurgical instrument is configured such that the first and the second jaws, when in the closed position, face proximally toward the distal end portion of the delivery shaft.
7. The electrosurgical instrument according to any one of Inventive Concepts 1 and 2, wherein the electrosurgical instrument is configured such that the first and the second jaws, when in the closed position, face distally way from the distal end portion of the delivery shaft.
8. The electrosurgical instrument according to any one of Inventive Concepts 1 and 2, wherein the electrically exposed cutting portion has a non-circular cross-section perpendicular to a longitudinal axis thereof.
9. The electrosurgical instrument according to Inventive Concept 8, wherein the non-circular cross-section has a shape selected from the group consisting of: a U-shape, a V-shape, a triangular shape, a diamond shape, and a horseshoe shape.
10. The electrosurgical instrument according to any one of Inventive Concepts 1 and 2, wherein the first and the second jaws are pivotally connected to each other.
11. The electrosurgical instrument according to any one of Inventive Concepts 1 and 2, wherein the first and the second jaws are slidingly connected to each other.
12. The electrosurgical instrument according to Inventive Concept 11, wherein the first and the second jaws are configured to slidingly move with respect to each other while respective planes defined by the first and the second leaflet-facing surfaces remain parallel to each other.
13. The electrosurgical instrument according to any one of Inventive Concepts 1 and 2, wherein one or both of the first and the second leaflet-facing surfaces are flat.
14. The electrosurgical instrument according to any one of Inventive Concepts 1 and 2, wherein one or both of the first and the second leaflet-facing surfaces are concave.
15. The electrosurgical instrument according to any one of Inventive Concepts 1 and 2, wherein the first and the second jaws include an electrically insulating material.
16. The electrosurgical instrument according to any one of Inventive Concepts 1 and 2, wherein at least one of the first and the second jaws includes a return electrode, which is configured to be coupled in electrical communication with the electrosurgical generator unit to provide bipolar electrical current between the electrically exposed cutting portion and the return electrode.
17. The electrosurgical instrument according to any one of Inventive Concepts 1 and 2, wherein at least one of the first and the second jaws includes one or more teeth that are configured to pierce the leaflet segment to be excised, when the first and the second jaws are in the closed position, so as to fixate the excised leaflet segment between the first and the second jaws.
18. The electrosurgical instrument according to any one of Inventive Concepts 1 and 2, wherein at least one of the first and the second jaws includes one or more teeth that are configured to grasp the leaflet segment to be excised, when the first and the second jaws are in the closed position, so as to fixate the excised leaflet segment between the first and the second jaws.
19. The electrosurgical instrument according to Inventive Concept 18, wherein the one or more teeth include 10 or more teeth disposed near a periphery of the at least one of the first and the second jaws.
20. The electrosurgical instrument according to any one of Inventive Concepts 1 and 2, wherein the electrosurgical instrument is configured such that the movement of the electrically exposed cutting portion along the predefined path excises the leaflet segment having a surface area of 3-60 mm2.
21. The electrosurgical instrument according to any one of Inventive Concepts 1 and 2, wherein one of the first and the second jaws is shaped so as to define a guidewire channel.
22. The electrosurgical instrument according to any one of Inventive Concepts 1 and 2, further including a channel having a distal opening near the first and the second jaws for delivery of a non-electrically-conductive liquid.
23. The electrosurgical instrument according to any one of Inventive Concepts 1-22,
   wherein the first jaw includes (a) a first outer shell, which includes an electrically-conductive metal; and (b) a first insert, which includes an electrically insulating material,
   wherein the first outer shell is shaped so as to define a first insert receptacle, which is shaped so as to receive the first insert, and the first insert is disposed within the first insert receptacle, and
   wherein the first insert is shaped so as to define at least a portion of the first leaflet-facing surface.
24. The electrosurgical instrument according to Inventive Concept 23,
   wherein the second jaw includes (a) a second outer shell, which includes an electrically-conductive metal; and (b) a second insert, which includes an electrically insulating material,
   wherein the second outer shell is shaped so as to define a second insert receptacle, which is shaped so as to receive the second insert, and the second insert is disposed within the second insert receptacle, and
   wherein the second insert is shaped so as to define at least a portion of the second leaflet-facing surface.
25. The electrosurgical instrument according to Inventive Concept 24,
   wherein the at least respective portions of the first and the second leaflet-facing surfaces defined by the first and the second inserts, respectively, are shaped so as to define respective first and second grooves, which together define the predefined path when the first and the second jaws are in the closed position,
   wherein, at least when the first and the second jaws are in the closed position, the first and the second electrically insulated wire portions are slidably disposed partially within the first and the second grooves, respectively, with the electrically exposed cutting portion extending between respective planes defined by the first and the second grooves, and
   wherein the electrosurgical instrument is configured such that when the first and the second jaws are in the closed position sandwiching the inserted leaflet portion and the electrical current is applied to the electrically exposed cutting portion by the electrosurgical generator unit, motion of the first and the second electrically insulated wire portions in the one or more longitudinal channels causes the movement of the electrically exposed cutting portion along the predefined path.
26. The electrosurgical instrument according to any one of Inventive Concepts 1-22, wherein the first electrically insulated wire portion, the electrically exposed cutting portion, and the second electrically insulated wire portion include three respective pieces fixed together, so as to define an elongate assembly.
27. The electrosurgical instrument according to Inventive Concept 26, wherein the electrically exposed cutting portion is shaped so as to define a sharp cutting surface.
28. The electrosurgical instrument according to any one of Inventive Concepts 1-22, wherein, at least when the first and the second jaws are in the closed position, the electrically exposed cutting portion includes a curved segment therealong.
29. The electrosurgical instrument according to Inventive Concept 28, wherein, at least when the first and the second jaws are in the closed position, the curved segment is semicircular.
30. The electrosurgical instrument according to any one of Inventive Concepts 1-22, wherein the electrosurgical instrument is configured such that the predefined path is not entirely straight.
31. The electrosurgical instrument according to Inventive Concept 30, wherein the electrosurgical instrument is configured such that the predefined path is U-shaped.
32. The electrosurgical instrument according to Inventive Concept 30, wherein the electrosurgical instrument is configured such that the predefined path includes one or more curved sections.
33. The electrosurgical instrument according to Inventive Concept 32, wherein the electrosurgical instrument is configured such that the predefined path includes exactly one curved section.
34. The electrosurgical instrument according to Inventive Concept 30, wherein the electrosurgical instrument is configured such that the predefined path includes one or more straight sections.
35. The electrosurgical instrument according to any one of Inventive Concepts 1-22,
   wherein the first and the second leaflet-facing surfaces are shaped so as to define respective first and second grooves, which together define the predefined path when the first and the second jaws are in the closed position,
   wherein, at least when the first and the second jaws are in the closed position, the first and the second electrically insulated wire portions are slidably disposed partially within the first and the second grooves, respectively, with the electrically exposed cutting portion extending between respective planes defined by the first and the second grooves, and
   wherein the electrosurgical instrument is configured such that when the first and the second jaws are in the closed position sandwiching the inserted leaflet portion and the electrical current is applied to the electrically exposed cutting portion by the electrosurgical generator unit, motion of the first and the second electrically insulated wire portions in the one or more longitudinal channels causes the movement of the electrically exposed cutting portion along the predefined path.
36. The electrosurgical instrument according to Inventive Concept 35,
   wherein the electrosurgical instrument includes a wire that includes the first and the second electrically insulated wire portions and, longitudinally therebetween, the electrically exposed cutting portion,
   wherein the first and the second grooves are shaped so as to define respective first and second longitudinal openings, which have respective widths that are narrower than greatest respective widths of the first and the second grooves,
   wherein respective widths of the wire at at least first and second longitudinal locations along the wire are greater than the respective widths of the first and the second longitudinal openings, and
   wherein a narrowest width of the electrically exposed cutting portion is less than the respective widths of the first and the second longitudinal openings.
37. The electrosurgical instrument according to Inventive Concept 35,
   wherein the electrosurgical instrument includes a wire that includes the first and the second electrically insulated wire portions and, longitudinally therebetween, the electrically exposed cutting portion,
   wherein the first and the second grooves are shaped so as to define respective first and second longitudinal openings, which have respective widths that are narrower than greatest respective widths of the first and the second grooves,
   wherein the wire includes first and second sliding beads fixed at first and second longitudinal locations along the wire,
   wherein the first and the second sliding beads are configured to slide through the first and the second grooves and have respective widths that are greater than the respective widths of the first and the second longitudinal openings, and
   wherein a narrowest width of the electrically exposed cutting portion is less than the respective widths of the first and the second sliding beads.
38. The electrosurgical instrument according to Inventive Concept 37, wherein the first and the second sliding beads include an electrically insulating material.
39. The electrosurgical instrument according to Inventive Concept 37, wherein the first and the second longitudinal locations are respectively located at (a) a first junction between the first electrically insulated wire portion and the electrically exposed cutting portion and (b) a second junction between the second electrically insulated wire portion and the electrically exposed cutting portion.
40. The electrosurgical instrument according to Inventive Concept 35,
   wherein the first electrically insulated wire portion, the electrically exposed cutting portion, and the second electrically insulated wire portion include three respective pieces fixed together, so as to define an elongate assembly,
   wherein the first and the second grooves are shaped so as to define respective first and second longitudinal openings, which have respective widths that are narrower than greatest respective widths of the first and the second grooves,
   wherein respective widths of the elongate assembly at at least first and second longitudinal locations along the elongate assembly are greater than the respective widths of the first and the second longitudinal openings, and
   wherein a narrowest width of the electrically exposed cutting portion is less than the respective widths of the first and the second longitudinal openings.
41. The electrosurgical instrument according to Inventive Concept 35,
   wherein the first electrically insulated wire portion, the electrically exposed cutting portion, and the second electrically insulated wire portion include three respective pieces fixed together, so as to define an elongate assembly,
   wherein the first and the second grooves are shaped so as to define respective first and second longitudinal openings, which have respective widths that are narrower than greatest respective widths of the first and the second grooves,
   wherein the elongate assembly includes first and second sliding beads fixed at first and second longitudinal locations along the elongate assembly,
   wherein the first and the second sliding beads are configured to slide through the first and the second grooves and have respective widths that are greater than the respective widths of the first and the second longitudinal openings, and
   wherein a narrowest width of the electrically exposed cutting portion is less than the respective widths of the first and the second sliding beads.
42. The electrosurgical instrument according to Inventive Concept 41, wherein the first and the second sliding beads include an electrically insulating material.
43. The electrosurgical instrument according to Inventive Concept 35, wherein the first and the second grooves are not entirely straight therealong.
44. The electrosurgical instrument according to Inventive Concept 43, wherein the first and the second grooves are U-shaped therealong.
45. The electrosurgical instrument according to Inventive Concept 43, wherein the first and the second grooves are shaped so as to define one or more corresponding curved sections, respectively.
46. The electrosurgical instrument according to Inventive Concept 45, wherein each of the first and the second grooves is shaped so as to define exactly one respective curved section.
47. The electrosurgical instrument according to Inventive Concept 43, wherein the first and the second grooves are shaped so as to define respective corresponding straight sections.
48. The electrosurgical instrument according to Inventive Concept 35, wherein the first and the second electrically insulated wire portions are slidably disposed partially within the first and the second grooves, respectively, both when the first and the second jaws are in the open position and are in the closed position.
49. The electrosurgical instrument according to Inventive Concept 35, wherein, at least when the first and the second jaws are in the closed position, the electrically exposed cutting portion includes a curved segment therealong.
50. The electrosurgical instrument according to Inventive Concept 49, wherein, at least when the first and the second jaws are in the closed position, the curved segment is semicircular.
51. The electrosurgical instrument according to Inventive Concept 35, wherein the electrosurgical instrument is configured such that when the first and the second jaws are in the closed position sandwiching the inserted leaflet portion and the electrical current is applied to the electrically exposed cutting portion by the electrosurgical generator unit, pulling of the first and the second electrically insulated wire portions in a proximal direction into the one or more longitudinal channels pulls the electrically exposed cutting portion along the predefined path.
52. The electrosurgical instrument according to Inventive Concept 51, wherein the electrosurgical instrument is configured such that when the first and the second jaws are in the closed position sandwiching the inserted leaflet portion and the electrical current is applied to the electrically exposed cutting portion by the electrosurgical generator unit, pulling of the first and the second electrically insulated wire portions in the proximal direction into the one or more longitudinal channels pulls the electrically exposed cutting portion along the predefined path such that the electrically exposed cutting portion first travels along the predefined path away from a junction between (a) the first and the second jaws and (b) the delivery shaft, and thereafter travels along the predefined path toward the junction.
53. The electrosurgical instrument according to Inventive Concept 52, wherein the electrosurgical instrument is configured such that the predefined path is U-shaped.
54. The electrosurgical instrument according to Inventive Concept 52, wherein the electrosurgical instrument is configured such that the predefined path includes one or more curved sections.
55. The electrosurgical instrument according to Inventive Concept 54, wherein the electrosurgical instrument is configured such that the predefined path includes exactly one curved section.
56. The electrosurgical instrument according to Inventive Concept 52, wherein the electrosurgical instrument is configured such that the predefined path includes one or more straight sections.
57. The electrosurgical instrument according to Inventive Concept 35, wherein the first and the second grooves are aligned with each other when the first and the second jaws are in the closed position.
58. The electrosurgical instrument according to Inventive Concept 35,
   wherein ends of the first groove are at an end of the first leaflet-facing surface that is supported at the distal end portion of the delivery shaft, and
   wherein ends of the second groove are at an end of the second leaflet-facing surface that is supported at the distal end portion of the delivery shaft.
59. The electrosurgical instrument according to Inventive Concept 35, wherein no portion of the electrically exposed cutting portion is within the first groove and no portion of the electrically exposed cutting portion is within the second groove.
60. A method of excising a segment of a native or prosthetic heart valve leaflet of a patient, the method including:
   electrically coupling, to an electrosurgical generator unit, an electrically exposed cutting portion of an electrosurgical instrument via at least one of first and second electrically insulated wire portions that longitudinal surround the electrically exposed cutting portion;
   advancing a delivery shaft of the electrosurgical instrument through vasculature of the patient to the native or prosthetic heart valve leaflet, wherein the delivery shaft is shaped so as to define one or more longitudinal channels therethrough, wherein the at least one of the first and the second electrically insulated wire portions passes through the one or more longitudinal channels, and wherein the delivery shaft supports, at a distal end portion thereof, first and second jaws that are shaped so as to define respective first and second leaflet-facing surfaces;
   while the first and second jaws are in an open position, in which the first and the second jaws are spaced apart, advancing the first and the second jaws so as to insert, between the first and the second jaws, a portion of the leaflet including the leaflet segment to be excised, such that the first and the second jaws are disposed on opposite sides of the leaflet;
   transitioning the first and the second jaws to a closed position, in which the first and the second jaws sandwich the inserted leaflet portion;
   thereafter, activating the electrosurgical generator unit to apply an electrical current to the electrically exposed cutting portion;
   excising the leaflet segment from the inserted leaflet portion by moving the electrically exposed cutting portion along a predefined path, such that the excised leaflet segment remains entrapped between the first and the second jaws; and
   thereafter, removing the electrosurgical instrument from the patient while the excised leaflet segment remains entrapped between the first and the second jaws.
61. The method according to Inventive Concept 60, wherein the electrosurgical instrument includes a wire that includes the first and the second electrically insulated wire portions and, longitudinally therebetween, the electrically exposed cutting portion.
62. The method according to Inventive Concept 60, wherein the native or prosthetic valve is a native or prosthetic aortic valve, and wherein excising includes excising the leaflet segment from the inserted leaflet portion of the native or prosthetic aortic valve.
63. The method according to Inventive Concept 60, wherein the native or prosthetic valve is a native mitral valve, and wherein excising includes excising the leaflet segment from the inserted leaflet portion of the native mitral valve.
64. The method according to Inventive Concept 60, further including, after excising the leaflet segment, implanting an artificial heart valve in the native or prosthetic heart valve.
65. The method according to Inventive Concept 60, wherein the electrosurgical instrument is configured such that the first and the second jaws, when in the closed position, face proximally toward the distal end portion of the delivery shaft.
66. The method according to Inventive Concept 60, wherein the electrosurgical instrument is configured such that the first and the second jaws, when in the closed position, face distally way from the distal end portion of the delivery shaft.
67. The method according to Inventive Concept 60, wherein transitioning the first and the second jaws to the closed position includes pivoting the first and the second jaws with respect to each other.
68. The method according to Inventive Concept 60, wherein transitioning the first and the second jaws to the closed position includes sliding the first and the second jaws closer to each other.
69. The method according to Inventive Concept 60, further including coupling a return electrode of at least one of the first and the second jaws in electrical communication with the electrosurgical generator unit, wherein activating the electrosurgical generator unit includes activating the electrosurgical generator unit to provide bipolar electrical current between the electrically exposed cutting portion and the return electrode.
70. The method according to Inventive Concept 60, wherein transitioning the first and the second jaws to the closed position includes piercing the leaflet segment to be excised with one or more teeth of at least one of the first and the second jaws, so as to fixate the excised leaflet segment between the first and the second jaws.
71. The method according to Inventive Concept 60, wherein transitioning the first and the second jaws to the closed position includes grasping the leaflet segment to be excised with one or more teeth of at least one of the first and the second jaws, so as to fixate the excised leaflet segment between the first and the second jaws.
72. The method according to Inventive Concept 71, wherein the one or more teeth include 10 or more teeth disposed near a periphery of the at least one of the first and the second jaws.
73. The method according to Inventive Concept 60, wherein excising the leaflet segment from the inserted leaflet portion includes excising the leaflet segment having a surface area of 3-60 mm2.
74. The method according to Inventive Concept 60, wherein advancing the delivery shaft through the vasculature includes:
   inserting, into the vasculature, a guidewire that passes through a guidewire channel defined by one of the first and the second jaws; and
   advancing the delivery shaft over the guidewire to the native or prosthetic heart valve.
75. The method according to Inventive Concept 60,
   wherein the first jaw includes (a) a first outer shell, which includes an electrically-conductive metal; and (b) a first insert, which includes an electrically insulating material,
   wherein the first outer shell is shaped so as to define a first insert receptacle, which is shaped so as to receive the first insert, and the first insert is disposed within the first insert receptacle, and
   wherein the first insert is shaped so as to define at least a portion of the first leaflet-facing surface.
76. The method according to Inventive Concept 75,
   wherein the second jaw includes (a) a second outer shell, which includes an electrically-conductive metal; and (b) a second insert, which includes an electrically insulating material,
   wherein the second outer shell is shaped so as to define a second insert receptacle, which is shaped so as to receive the second insert, and the second insert is disposed within the second insert receptacle, and
   wherein the second insert is shaped so as to define at least a portion of the second leaflet-facing surface.
77. The method according to Inventive Concept 76,
   wherein the at least respective portions of the first and the second leaflet-facing surfaces defined by the first and the second inserts, respectively, are shaped so as to define respective first and second grooves, which together define the predefined path when the first and the second jaws are in the closed position,
   wherein, at least when the first and the second jaws are in the closed position, the first and the second electrically insulated wire portions are slidably disposed partially within the first and the second grooves, respectively, with the electrically exposed cutting portion extending between respective planes defined by the first and the second grooves, and
   wherein excising the leaflet segment from the inserted leaflet portion includes moving the electrically exposed cutting portion along the predefined path by moving the first and the second electrically insulated wire portions in the one or more longitudinal channels.
78. The method according to Inventive Concept 60, wherein the first electrically insulated wire portion, the electrically exposed cutting portion, and the second electrically insulated wire portion include three respective pieces fixed together, so as to define an elongate assembly.
79. The method according to Inventive Concept 78, wherein the electrically exposed cutting portion is shaped so as to define a sharp cutting surface.
80. The method according to Inventive Concept 60, wherein the electrosurgical instrument is configured such that the predefined path is not entirely straight.
81. The method according to Inventive Concept 80, wherein the electrosurgical instrument is configured such that the predefined path is U-shaped.
82. The method according to Inventive Concept 80, wherein the electrosurgical instrument is configured such that the predefined path includes one or more curved sections.
83. The method according to Inventive Concept 82, wherein the electrosurgical instrument is configured such that the predefined path includes exactly one curved section.
84. The method according to Inventive Concept 80, wherein the electrosurgical instrument is configured such that the predefined path includes one or more straight sections.
85. The method according to Inventive Concept 60,
   wherein the first and the second leaflet-facing surfaces are shaped so as to define respective first and second grooves, which together define the predefined path when the first and the second jaws are in the closed position,
   wherein, at least when the first and the second jaws are in the closed position, the first and the second electrically insulated wire portions are slidably disposed partially within the first and the second grooves, respectively, with the electrically exposed cutting portion extending between respective planes defined by the first and the second grooves, and
   wherein excising the leaflet segment from the inserted leaflet portion includes moving the electrically exposed cutting portion along the predefined path by moving the first and the second electrically insulated wire portions in the one or more longitudinal channels.
86. The method according to Inventive Concept 85,
   wherein the electrosurgical instrument includes a wire that includes the first and the second electrically insulated wire portions and, longitudinally therebetween, the electrically exposed cutting portion,
   wherein the first and the second grooves are shaped so as to define respective first and second longitudinal openings, which have respective widths that are narrower than greatest respective widths of the first and the second grooves,
   wherein respective widths of the wire at at least first and second longitudinal locations along the wire are greater than the respective widths of the first and the second longitudinal openings, and
   wherein a narrowest width of the electrically exposed cutting portion is less than the respective widths of the first and the second longitudinal openings.
87. The method according to Inventive Concept 85,
   wherein the electrosurgical instrument includes a wire that includes the first and the second electrically insulated wire portions and, longitudinally therebetween, the electrically exposed cutting portion,
   wherein the first and the second grooves are shaped so as to define respective first and second longitudinal openings, which have respective widths that are narrower than greatest respective widths of the first and the second grooves,
   wherein the wire includes first and second sliding beads fixed at first and second longitudinal locations along the wire,
   wherein the first and the second sliding beads are configured to slide through the first and the second grooves and have respective widths that are greater than the respective widths of the first and the second longitudinal openings, and
   wherein a narrowest width of the electrically exposed cutting portion is less than the respective widths of the first and the second sliding beads.
88. The method according to Inventive Concept 87, wherein the first and the second longitudinal locations along the wire are respectively located at (a) a first junction between the first electrically insulated wire portion and the electrically exposed cutting portion and (b) a second junction between the second electrically insulated wire portion and the electrically exposed cutting portion.
89. The method according to Inventive Concept 85,
   wherein the first electrically insulated wire portion, the electrically exposed cutting portion, and the second electrically insulated wire portion include three respective pieces fixed together, so as to define an elongate assembly,
   wherein the first and the second grooves are shaped so as to define respective first and second longitudinal openings, which have respective widths that are narrower than greatest respective widths of the first and the second grooves,
   wherein respective widths of the elongate assembly at at least first and second longitudinal locations along the elongate assembly are greater than the respective widths of the first and the second longitudinal openings, and
   wherein a narrowest width of the electrically exposed cutting portion is less than the respective widths of the first and the second longitudinal openings.
90. The method according to Inventive Concept 85,
   wherein the first electrically insulated wire portion, the electrically exposed cutting portion, and the second electrically insulated wire portion include three respective pieces fixed together, so as to define an elongate assembly,
   wherein the first and the second grooves are shaped so as to define respective first and second longitudinal openings, which have respective widths that are narrower than greatest respective widths of the first and the second grooves,
   wherein the elongate assembly includes first and second sliding beads fixed at first and second longitudinal locations along the elongate assembly,
   wherein the first and the second sliding beads are configured to slide through the first and the second grooves and have respective widths that are greater than the respective widths of the first and the second longitudinal openings, and
   wherein a narrowest width of the electrically exposed cutting portion is less than the respective widths of the first and the second sliding beads.
91. The method according to Inventive Concept 85, wherein the first and the second grooves are not entirely straight therealong.
92. The method according to Inventive Concept 91, wherein the first and the second grooves are U-shaped therealong.
93. The method according to Inventive Concept 91, wherein the first and the second grooves are shaped so as to define one or more corresponding curved sections, respectively.
94. The method according to Inventive Concept 93, wherein each of the first and the second grooves is shaped so as to define exactly one respective curved section.
95. The method according to Inventive Concept 91, wherein the first and the second grooves are shaped so as to define respective corresponding straight sections.
96. The method according to Inventive Concept 85, wherein the first and the second electrically insulated wire portions are slidably disposed partially within the first and the second grooves, respectively, both when the first and the second jaws are in the open position and are in the closed position.
97. The method according to Inventive Concept 85, wherein, at least when the first and the second jaws are in the closed position, the electrically exposed cutting portion includes a curved segment therealong.
98. The method according to Inventive Concept 97, wherein, at least when the first and the second jaws are in the closed position, the curved segment is semicircular.
99. The method according to Inventive Concept 85, wherein moving the electrically exposed cutting portion along the predefined path by moving the first and the second electrically insulated wire portions in the one or more longitudinal channels includes:
   pulling the electrically exposed cutting portion along the predefined path by pulling the first and the second electrically insulated wire portions in a proximal direction into the one or more longitudinal channels.
100. The method according to Inventive Concept 99, wherein the electrosurgical instrument is configured such that when the first and the second jaws are in the closed position sandwiching the inserted leaflet portion and the electrical current is applied to the electrically exposed cutting portion by the electrosurgical generator unit, pulling of the first and the second electrically insulated wire portions in the proximal direction into the one or more longitudinal channels pulls the electrically exposed cutting portion along the predefined path such that the electrically exposed cutting portion first travels along the predefined path away from a junction between (a) the first and the second jaws and (b) the delivery shaft, and thereafter travels along the predefined path toward the junction.
101. The method according to Inventive Concept 100, wherein the electrosurgical instrument is configured such that the predefined path is U-shaped.
102. The method according to Inventive Concept 100, wherein the electrosurgical instrument is configured such that the predefined path includes one or more curved sections.
103. The method according to Inventive Concept 102, wherein the electrosurgical instrument is configured such that the predefined path includes exactly one curved section.
104. The method according to Inventive Concept 100, wherein the electrosurgical instrument is configured such that the predefined path includes one or more straight sections.
105. The method according to Inventive Concept 85, wherein the first and the second grooves are aligned with each other when the first and the second jaws are in the closed position.
106. The method according to Inventive Concept 85,
   wherein ends of the first groove are at an end of the first leaflet-facing surface that is supported at the distal end portion of the delivery shaft, and
   wherein ends of the second groove are at an end of the second leaflet-facing surface that is supported at the distal end portion of the delivery shaft.
107. The method according to Inventive Concept 85, wherein no portion of the electrically exposed cutting portion is within the first groove and no portion of the electrically exposed cutting portion is within the second groove.
108. The method according to Inventive Concept 60, further including delivering a non-electrically-conductive liquid via a channel having a distal opening near the first and the second jaws, so as to dilute blood in a vicinity of the first and the second jaws.

The present invention will be more fully understood from the following detailed description of embodiments thereof, taken together with the drawings, in which:

### BRIEF DESCRIPTION OF THE DRAWINGS

FIG. 1 is a schematic illustration of an electrosurgical instrument for use with an electrosurgical generator unit, for excising a segment of a native or prosthetic heart valve leaflet, in accordance with an application of the present invention;

FIGS. 2A-B are schematic illustrations of a portion of the electrosurgical instrument of FIG. 1 with first and second jaws thereof in a closed position, in accordance with an application of the present invention;

FIGS. 3A-B are schematic cross-sectional illustrations of a portion of the electrosurgical instrument of FIG. 1 taken along lines IIIA-IIIA and IIIB-IIIB of FIG. 2B, respectively, with the first and the second jaws in the closed position, in accordance with an application of the present invention;

FIGS. 4A-C are schematic illustrations of a portion of the electrosurgical instrument of FIG. 1 with the first and the second jaws pivotally connected to each other and in an open position, in accordance with an application of the present invention;

FIGS. 4D and 4E-F are schematic isometric and cross-sectional illustrations of another configuration of the first and the second jaws of the electrosurgical instrument of FIG. 1, in accordance with an application of the present invention;

FIGS. 5A-C are schematic illustrations of a portion of the electrosurgical instrument of FIG. 1 with the first and the second jaws in the closed position with an electrically exposed cutting portion at three respective different locations along a predefined path, in accordance with an application of the present invention;

FIG. 6 is a schematic illustration of a portion of another electrosurgical instrument in which first and second jaws thereof are slidingly connected to each other, in accordance with an application of the present invention;

FIG. 7 is a schematic illustration of a portion of a bipolar electrosurgical instrument with first and second jaws thereof in an open position, in accordance with an application of the present invention;

FIGS. 8A-G are schematic illustrations of a method of excising a segment of a native or prosthetic heart valve leaflet of a patient, in accordance with an application of the present invention;

FIGS. 9A-B are schematic illustrations of the leaflet of FIGS. 8A-G after it has been excised using the electrosurgical instrument of FIG. 1 as described with reference to FIGS. 8A-G, in accordance with an application of the present invention;

FIG. 10 is a schematic illustration of a portion of another electrosurgical instrument and a method of using the electrosurgical instrument with an electrosurgical generator unit to excise a segment of a mitral valve leaflet;

FIG. 11 is a schematic illustration of an operating handle positioned at a distal end of the electrosurgical instrument of FIG. 1, in accordance with an application of the present invention;

FIGS. 12A-B are schematic illustrations of a portion of yet another electrosurgical instrument, in accordance with an application of the present invention;

FIGS. 13A-B are schematic illustrations of respective configurations of a portion of still another electrosurgical instrument, in accordance with respective applications of the present invention; and

FIG. 14 includes several photographs of a prototype and experiments conducted by the inventors, in accordance with an application of the present invention.

### DETAILED DESCRIPTION OF THE APPLICATION

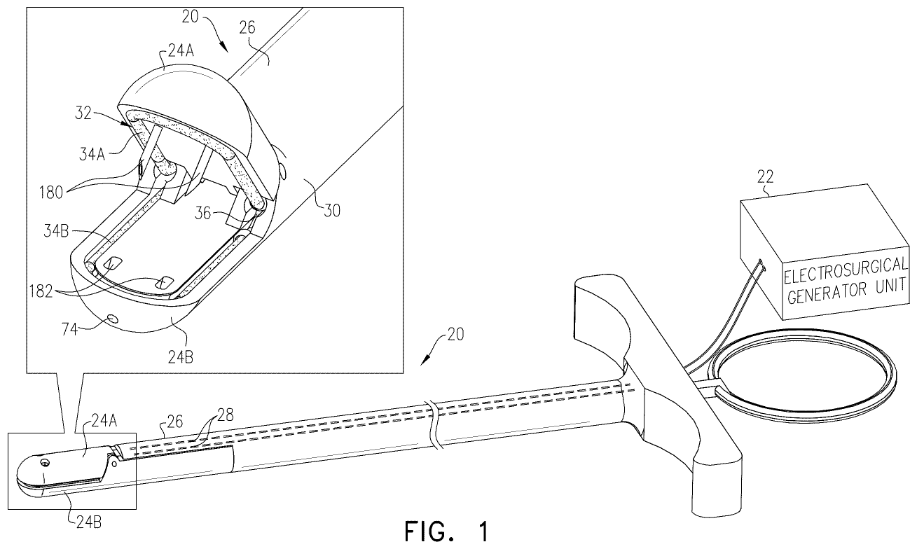

Reference is made to FIG. 1, which is a schematic illustration of an electrosurgical instrument 20 for use with an electrosurgical generator unit 22, for excising a segment of a native or prosthetic heart valve leaflet, in accordance with an application of the present invention.

Electrosurgical generator unit 22 is typically not an element of the invention, and may be a conventional electrosurgical generator unit configured to be operated as described hereinbelow. As is known in the art, electrosurgical generator units (ESUs) supply high-frequency alternating electrical current to cut or coagulate tissue during electrosurgery. For some applications, electrosurgical generator unit 22 is configured to apply the current in a pure cut mode, as is known in the electrosurgical and electrocautery arts. Pure cut mode produces high amplitude continuous waves, as is known in the art. Typically, electrosurgical generator unit 22 is activated at 30-100 Watts and/or at 200-500 kHz, such as, 250-450 kHz.

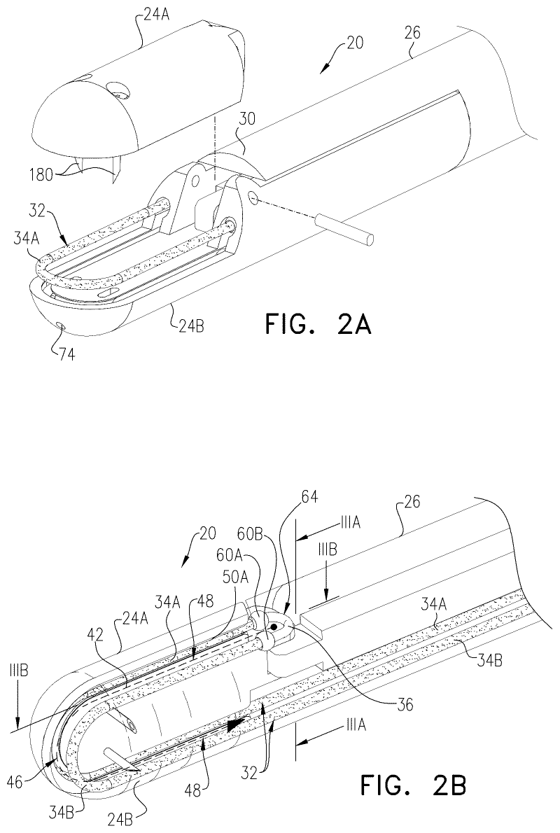

Reference is also made to FIGS. 2A-B, which are schematic illustrations of a portion of electrosurgical instrument 20 with first and second jaws 24A and 24B thereof in a closed position, in accordance with an application of the present invention. FIG. 2A is an exploded view. For clarity of illustration, second jaw 24B is shown as transparent in FIG. 2B.

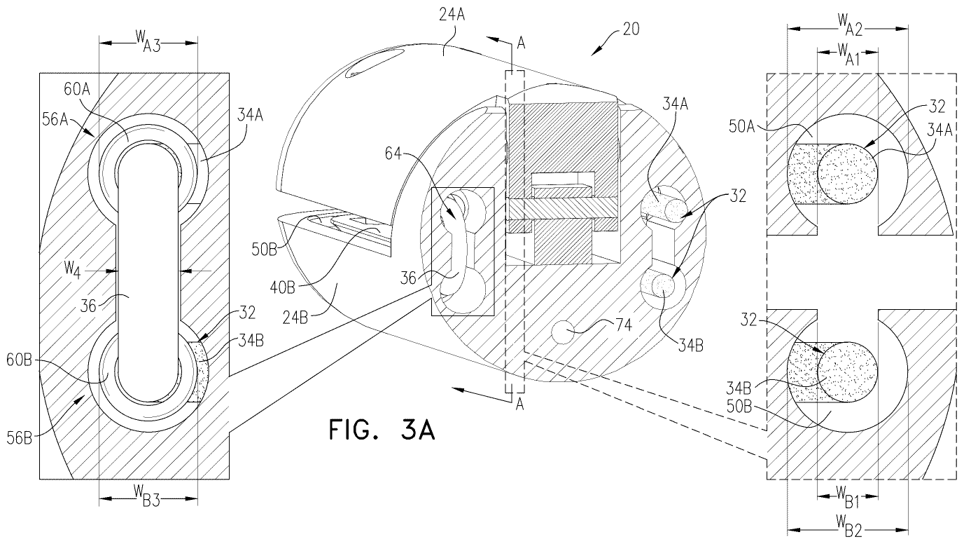

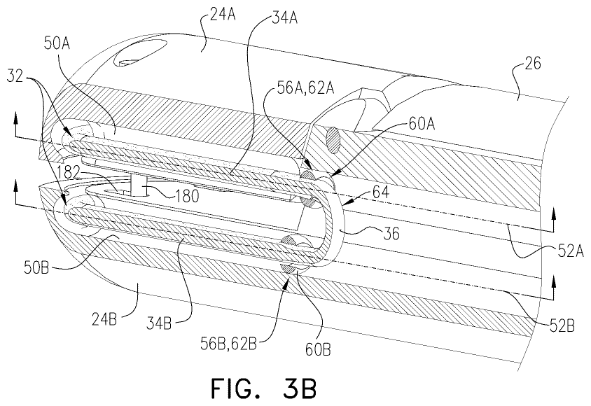

Reference is further made to FIGS. 3A-B, which are schematic cross-sectional illustrations of a portion of electrosurgical instrument 20 taken along lines IIIA-IIIA and IIIB-IIIB of FIG. 2B, respectively, with first and second jaws 24A and 24B in the closed position, in accordance with an application of the present invention.

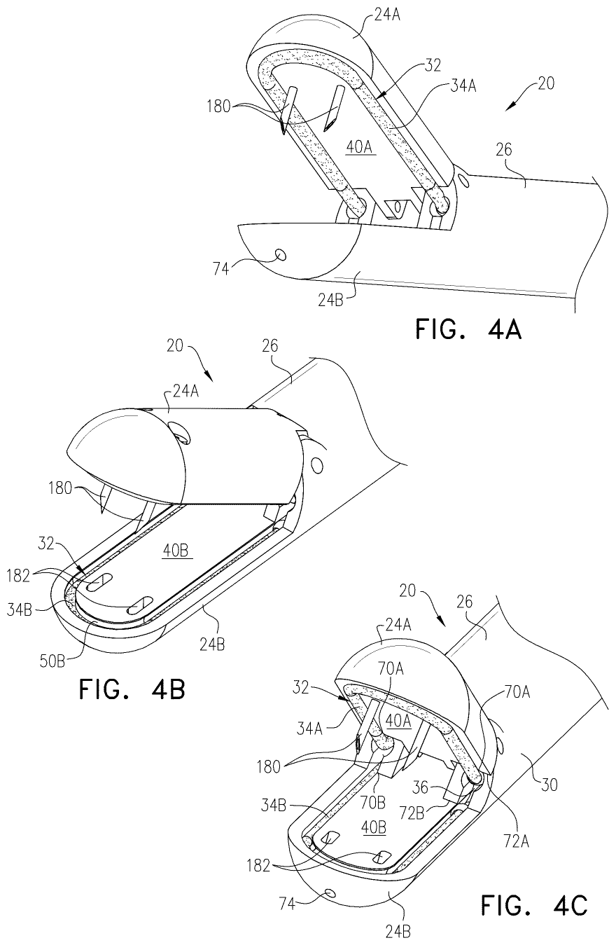

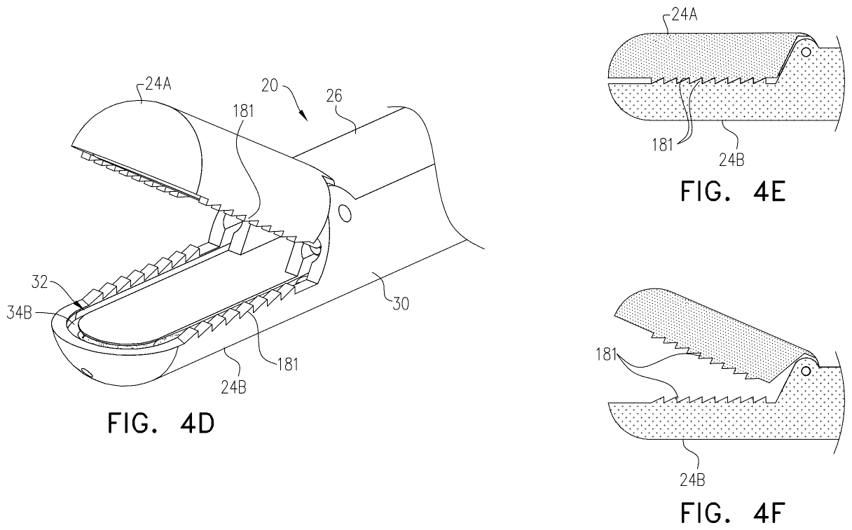

Reference is still further made to FIGS. 4A-C, which are schematic illustrations of a portion of electrosurgical instrument 20 with first and second jaws 24A and 24B thereof in an open position, in accordance with an application of the present invention.

Electrosurgical instrument 20 comprises:
a delivery shaft 26, which is shaped so as to define one or more longitudinal channels 28 therethrough (shown in FIG. 1);
first and second jaws 24A and 24B, which are supported at a distal end portion 30 of delivery shaft 26; and
first and second electrically insulated wire portions 34A and 34B and, longitudinally therebetween, an electrically exposed cutting portion 36.

First and second electrically insulated wire portions 34A and 34B comprise respective conductive wires coated and/or encased in an electrically-insulating material. For some applications, electrically-insulating material of first and second electrically insulated wire portions 34A and 34B comprises a polymer, typically a heat-resistance polymer, such as polyimide or polytetrafluoroethylene (PTFE).

For some applications, electrosurgical instrument 20 comprises a wire 32 that comprises first and second electrically insulated wire portions 34A and 34B and, longitudinally therebetween, electrically exposed cutting portion 36. For some of these applications, wire 32 comprises a single integral elongate piece of metal that defines first and second electrically insulated wire portions 34A and 34B and electrically exposed cutting portion 36.

For other applications, first electrically insulated wire portion 34A, electrically exposed cutting portion 36, and second electrically insulated wire portion 34B comprise three respective pieces fixed together (by an electrical and mechanical bonding/connection), which collectively define an elongate assembly. In these latter applications, electrically exposed cutting portion 36 may have a different diameter than the insulated wire portions, and/or may comprise a different type of metal than the insulated wire portions.

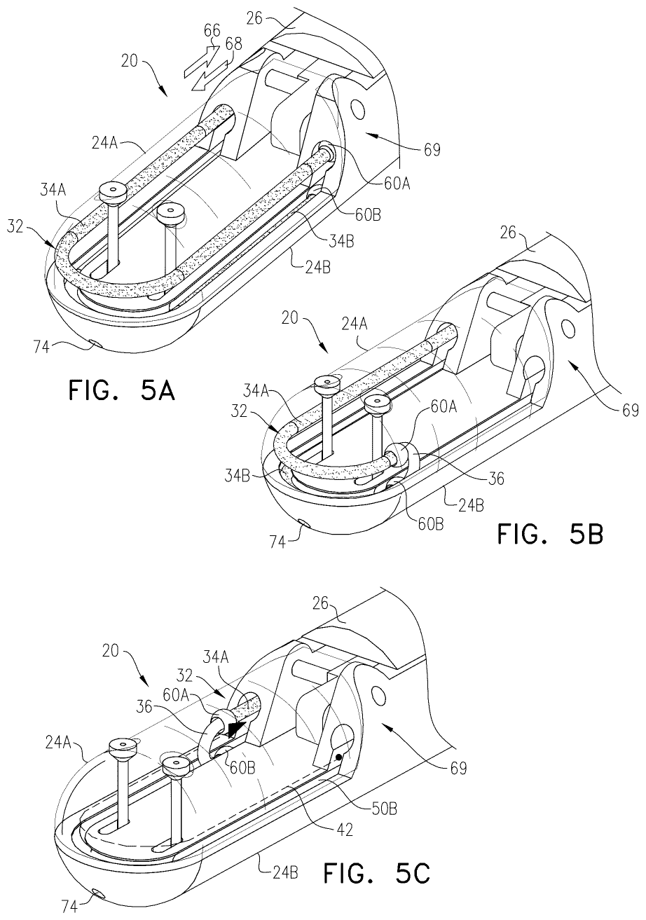

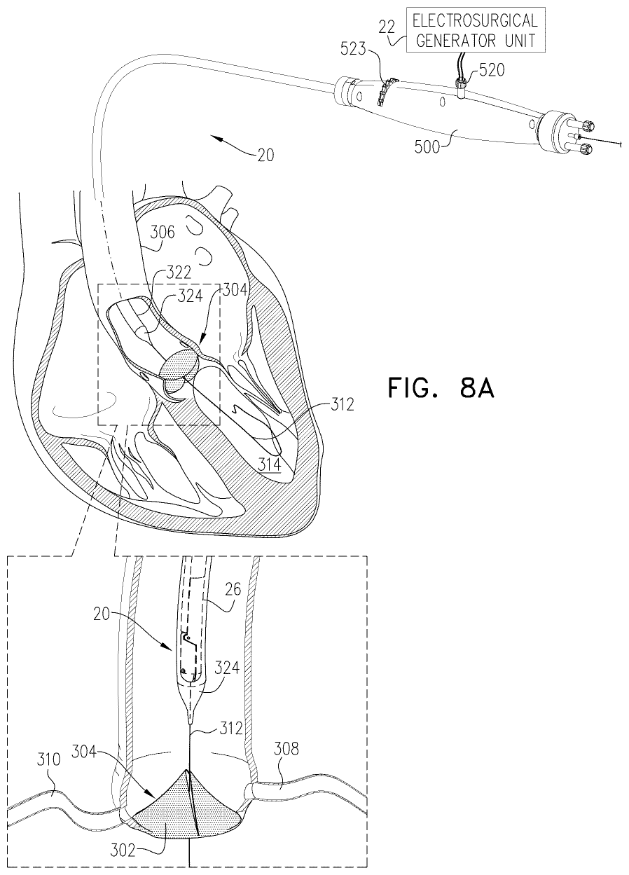

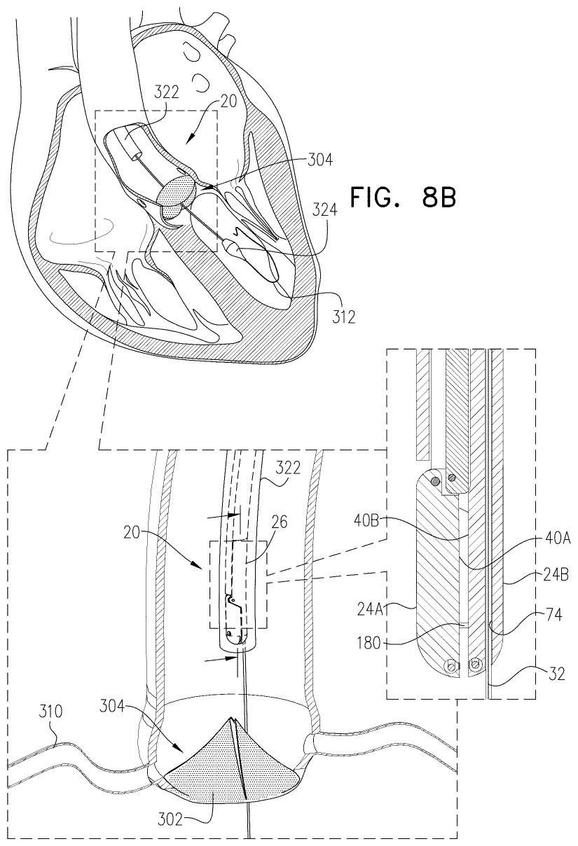

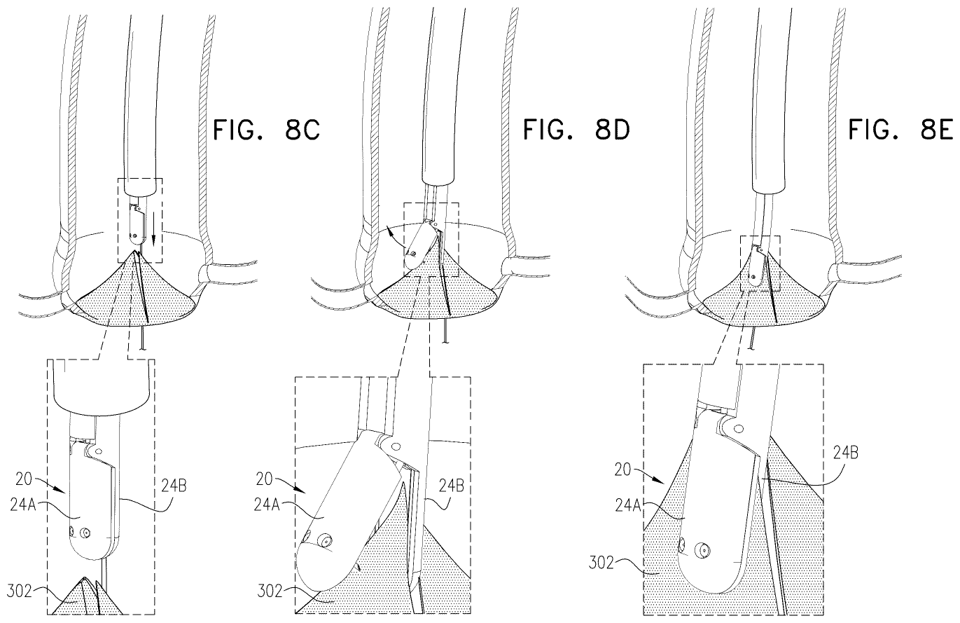

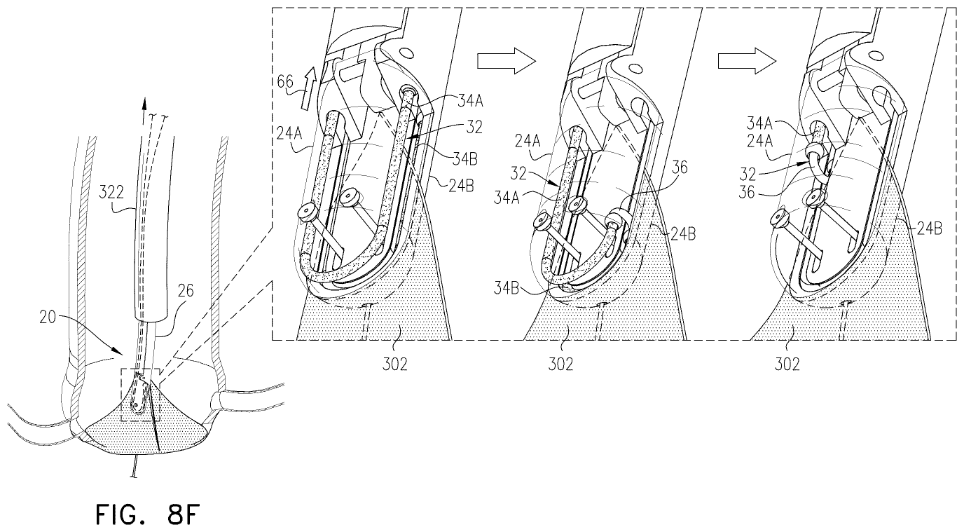

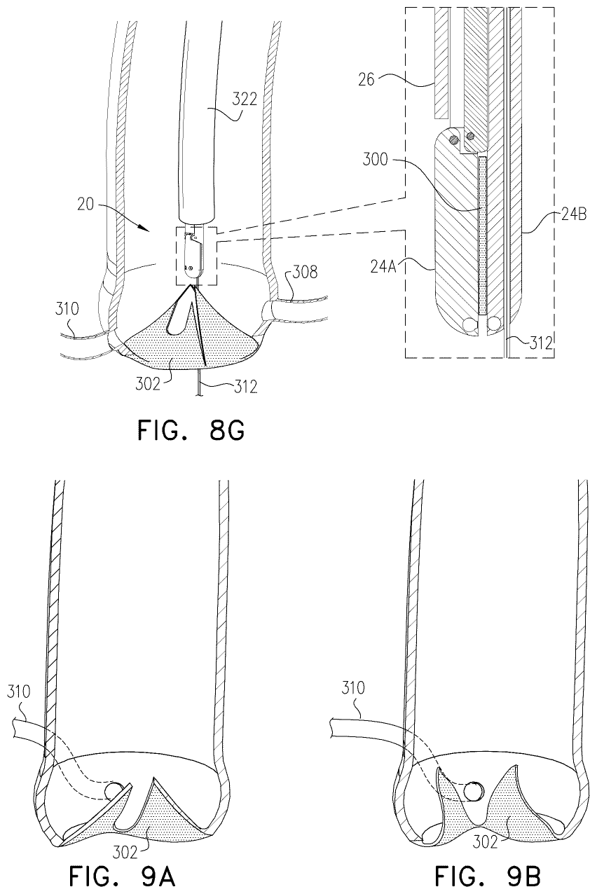

First and second jaws 24A and 24B are shaped so as to define respective first and second leaflet-facing surfaces 40A and 40B (labeled in FIGS. 4A-C and 8B). First and second jaws 24A and 24B together are configured to assume:
the open position, in which first and second jaws 24A and 24B are spaced apart to allow insertion, between first and second jaws 24A and 24B, of a portion of the leaflet including the leaflet segment to be excised, such as shown in FIGS. 4A-C, as well as in FIG. 8D, described hereinbelow; and
the closed position, in which first and second jaws 24A and 24B sandwich the inserted leaflet portion, such as shown in FIGS. 1, 2A-B, and 3A-B, as well as FIGS. 5A-C, 8B-C, and 8E-G, described hereinbelow.

At least one (optionally both) of first and second electrically insulated wire portions 34A and 34B passes through the one or more longitudinal channels 28 of delivery shaft 26 and is configured to couple electrically exposed cutting portion 36 in electrical communication with electrosurgical generator unit 22.

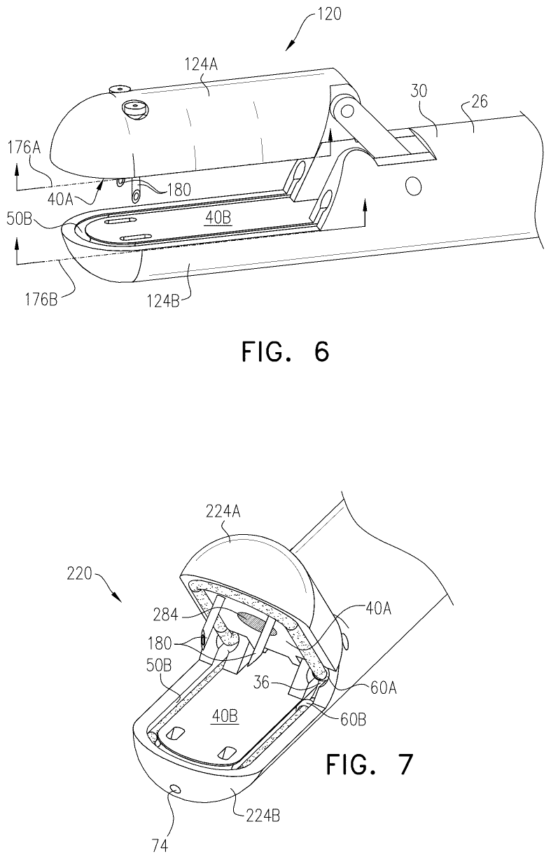

For some applications, first and second jaws 24A and 24B are pivotally (hingedly) connected to each other, such as shown in FIGS. 1-4C. For other applications, first and second jaws 24A and 24B are slidingly connected to each other, such as described hereinbelow with reference to FIG. 6. In either case, an orientation of one of the jaws (e.g., second jaw 24B) may be fixed with respect to distal end portion 30 of delivery shaft 26, while the other jaw (e.g., first jaw 24A) may be moveable with distal end portion 30.

For some applications, one or both of first and second leaflet-facing surfaces 40A and 40B are flat, such as shown in the figures. For other applications, one or both of first and second leaflet-facing surfaces 40A and 40B are concave, in which case edges of leaflet-facing surfaces grasp the inserted leaflet portion (configuration not shown).

Typically, first and second jaws 24A and 24B comprise an electrically insulating material, such that the current applied using electrosurgical generator unit 22 is directed through tissue of the inserted leaflet portion, rather than through material of the jaws. For example, the electrically insulating material may comprise (a) a polymer, such as polyimide, polyether ether ketone (PEEK), polyethersulfone (PES), or polytetrafluoroethylene (PTFE); or (b) a bioceramic material, (c) a bioglass material, or (d) vitreous carbon.

For some applications, the wire of first and second electrically insulated wire portions 34A and 34B (and/or wire 32, if provided) comprise tungsten, Nitinol, or stainless steel. These metals are excellent electrical conductors, can withstand the high temperatures necessary for tissue cutting, and have good mechanical strength for pulling of the wire, such as described hereinbelow with reference to FIGS. 5A-C. The wire of first and second electrically insulated wire portions 34A and 34B (and/or wire 32, if provided) may also comprise other metals, such as silver.

For some applications, each wire of first and second electrically insulated wire portions 34A and 34B (and/or wire 32, if provided) comprises a plurality of wires coupled to one another, such as interwoven so as to form a cable.

For some applications, each wire of first and second electrically insulated wire portions 34A and 34B (and/or wire 32, if provided) comprises a first wire coupled to a plurality of second wires, such as interwoven so as to form a cable. For example, the first wire may be coupled to the plurality of second wires by soldering or crimping. The first wire and the second wires may comprise the same or different materials, typically metals.

Reference is still made to FIGS. 1, 2A-B, 3A-B, and 4A-C, and is additionally made to FIGS. 5A-C, which are schematic illustrations of a portion of electrosurgical instrument 20 with first and second jaws 24A and 24B in the closed position, in accordance with an application of the present invention. For clarity of illustration, first jaw 24A is shown as transparent in FIGS. 5A-C (and in FIG. 8F, described hereinbelow).

Electrosurgical instrument 20 is configured such that when first and second jaws 24A and 24B are in the closed position sandwiching the inserted leaflet portion and an electrical current is applied to electrically exposed cutting portion 36 by electrosurgical generator unit 22, movement of electrically exposed cutting portion 36 along a predefined path 42 (labeled in FIGS. 2B and 5C) excises the leaflet segment from the inserted leaflet portion, such that the excised leaflet segment remains entrapped between first and second jaws 24A and 24B.

FIGS. 5A-C show electrically exposed cutting portion 36 at three respective different locations along predefined path 42. For some applications, the location shown in FIG. 5A may be considered an initial, or starting, location, at which electrically exposed cutting portion 36 is disposed at the beginning of the procedure.

Reference is made to FIGS. 2A and 5A-C. Electrosurgical instrument 20 is typically configured such that predefined path 42 is not entirely straight. Typically, electrosurgical instrument 20 is configured such that predefined path 42 includes one or more curved sections 46, such as exactly one curved section 46, such as shown (labeled in FIG. 2B). Typically, electrosurgical instrument 20 is configured such that predefined path 42 further includes one or more straight sections 48, e.g., two straight sections 48, such as shown (labeled in FIG. 2B). For example, electrosurgical instrument 20 may be configured such that predefined path 42 is U-shaped, such as shown.

Reference is made to FIGS. 2A-B, 3A-B, 4A-C, and 5A-C. For some applications, first and second leaflet-facing surfaces 40A and 40B are shaped so as to define respective first and second grooves 50A and 50B (i.e., elongate indentations), which together define predefined path 42 when first and second jaws 24A and 24B are in the closed position. At least when first and second jaws 24A and 24B are in the closed position, first and second electrically insulated wire portions 34A and 34B are slidably disposed partially within first and second grooves 50A and 50B, respectively, with electrically exposed cutting portion 36 extending between respective planes 52A and 52B defined by first and second grooves 50A and 50B (labeled in FIG. 3B).

Electrosurgical instrument 20 is configured such that when first and second jaws 24A and 24B are in the closed position sandwiching the inserted leaflet portion and the electrical current is applied to electrically exposed cutting portion 36 by electrosurgical generator unit 22, motion of first and second electrically insulated wire portions 34A and 34B in the one or more longitudinal channels 28 causes the movement of electrically exposed cutting portion 36 along predefined path 42.

Thus, first and second electrically insulated wire portions 34A and 34B serve two functions:
conducting the current to electrically exposed cutting portion 36 (electrosurgical instrument 20 may be configured such that one or both of first and second electrically insulated wire portions 34A and 34B conduct the current to electrically exposed cutting portion 36); and
remotely conveying a mechanical force to electrically exposed cutting portion 36 to move the cutting portion.

The motion of first and second electrically insulated wire portions 34A and 34B may be caused manually by a human operator. Alternatively, electrosurgical instrument 20 may comprise a motor that is configured to cause the motion.

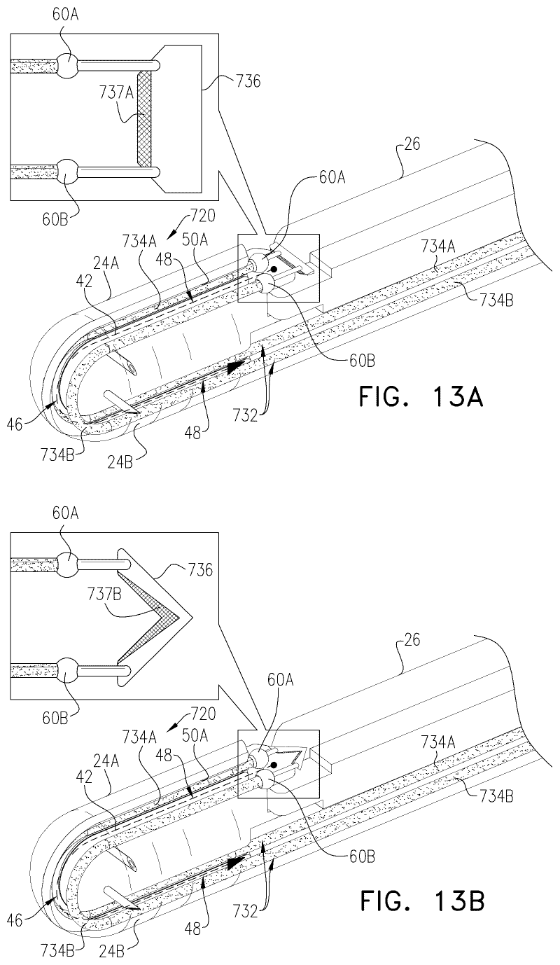

For some of these applications, as labeled in FIG. 3A, first and second grooves 50A and 50B are shaped so as to define respective first and second longitudinal openings 54A and 54B, which have respective widths W_{A1} and W_{B1} that are narrower than greatest respective widths W_{A2} and W_{B2} of first and second grooves 50A and 50B. For applications in which electrosurgical instrument 20 comprises wire 32, respective widths W_{A3} and W_{B3} of wire 32 at at least first and second longitudinal locations 56A and 56B along wire 32 are greater than the respective widths W_{A1} and W_{B1} of first and second longitudinal openings 54A and 54B. For applications in which electrosurgical instrument 20 comprises the elongate assembly described above or elongate assembly 732 described hereinbelow with reference to FIGS. 13A-B, respective widths W_{A3} and W_{B3} of the elongate assembly at at least first and second longitudinal locations 56A and 56B along the elongate assembly are greater than the respective widths W_{A1} and W_{B1} of first and second longitudinal openings 54A and 54B.

A narrowest width W₄ of electrically exposed cutting portion 36 is less than the respective widths W_{A1} and W_{B1} of first and second longitudinal openings 54A and 54B. As a result, electrically exposed cutting portion 36 can move along predefined path 42 defined by first and second grooves 50A and 50B, between the respective planes defined by the grooves, and first and second electrically insulated wire portions 34A and 34B do not become detached from first and second grooves 50A and 50B as electrically exposed cutting portion 36 moves along predefined path 42. First and second longitudinal locations 56A and 56B are typically along first and second electrically insulated wire portions 34A and 34B.

(Widths W_{A1} and W_{B1} and widths W_{A2} and W_{B2} are measured perpendicular to a longitudinal axis of first and second grooves 50A and 50B. Widths W_{A3} and W_{B3} of wire 32 or the elongate assembly are measured perpendicular to a longitudinal axis of wire 32 or the elongate assembly, as the case may be.)

For some applications, first and second grooves 50A and 50B have a cross-section perpendicular to a longitudinal axis thereof that is shaped as a partial circle (i.e., a C), a T, or any other configuration.

For some applications in which electrosurgical instrument 20 comprises wire 32, wire 32 comprises first and second sliding beads 60A and 60B fixed at first and second longitudinal locations 56A and 56B along wire 32. For some applications in which electrosurgical instrument 20 comprises the elongate assembly described above or elongate assembly 732 described hereinbelow with reference to FIGS. 13A-B, the elongate assembly comprises first and second sliding beads 60A and 60B fixed at first and second longitudinal locations 56A and 56B along the elongate assembly.

Optionally, electrosurgical instrument 20 comprises more than two beads, such as an even number of beads, e.g., 4 beads.

First and second sliding beads 60A and 60B have respective widths W_{A3} and W_{B3}. First and second sliding beads 60A and 60B are configured to slide through first and second grooves 50A and 50B. For example, a shape of first and second sliding beads 60A and 60B, in cross-section perpendicular to a longitudinal axis of wire 32 or the elongate assembly, as the base may be, may be circular (as shown), elliptical, polygonal (e.g., square, triangular, or star-shaped) (configurations not shown).

For some applications, first and second sliding beads 60A and 60B comprise an electrically insulating material, such that the current applied using electrosurgical generator unit 22 is directed through tissue of the inserted leaflet portion only via electrically exposed cutting portion 36, rather than also via first and second sliding beads 60A and 60B.

For some applications, such as labeled in FIG. 3B, first and second longitudinal locations 56A and 56B along wire 32 or the elongate assembly are respectively located at (a) a first junction 62A between first electrically insulated wire portion 34A and electrically exposed cutting portion 36 and (b) a second junction 62B between second electrically insulated wire portion 34B and electrically exposed cutting portion 36.

Reference is made to FIG. 1A. First and second grooves 50A and 50B are typically not entirely straight therealong, so as to define predefined path 42 that is not entirely straight, as described hereinabove with reference to FIG. 1A. Typically, first and second grooves 50A and 50B are shaped so as to define one or more corresponding curved sections, respectively, so as to define the one or more curved sections 46 of predefined path 42, as described hereinabove with reference to FIG. 1A; for example, each of first and second grooves 50A and 50B may be shaped so as to define exactly one respective curved section. Typically, first and second grooves 50A and 50B are shaped so as to define respective corresponding straight sections, so as to define the one or more straight sections 48 of predefined path 42, as described hereinabove with reference to FIG. 1A. For example, first and second grooves 50A and 50B may be U-shaped therealong (i.e., the U-shapes are defined by the longitudinal shape of the grooves, as opposed to cross-sections of the grooves perpendicular to their respective longitudinal axes), so as to define the U-shape of predefined path 42, as described hereinabove with reference to FIG. 1A.

Reference is again made to FIGS. 2A-B, 3A-B, and 4A-C. For some applications, first and second electrically insulated wire portions 34A and 34B are slidably disposed partially within first and second grooves 50A and 50B, respectively, both when first and second jaws 24A and 24B (a) are in the open position, such as shown in FIGS. 4A-C, as well as in FIG. 8D, described hereinbelow, and (b) are in the closed position, such as shown in FIGS. 1, 2A-B, 3A-B, and 5A-C, as well as FIGS. 8B-C and 8E-G, described hereinbelow.

Reference is made to FIGS. 2B and 3A-B. For some applications, at least when first and second jaws 24A and 24B are in the closed position, electrically exposed cutting portion 36 includes a curved segment 64 therealong (i.e., curved segment 64 is defined by the longitudinal shape of the electrically exposed cutting portion, as opposed to a cross-section of the electrically exposed cutting portion perpendicular to its longitudinal axis). Curved segment 64 may allow simultaneous cutting of the leaflet from both sides of the leaflet, because respective portions of the curve touch opposing surfaces of the leaflet. For some of these applications, at least when first and second jaws 24A and 24B are in the closed position, curved segment 64 is semicircular, such as shown in the figures.

Reference is still made to FIGS. 2B and 3A-B and is additionally made to FIG. 4C. For some applications, electrically exposed cutting portion 36 includes curved segment 64 therealong both when first and second jaws 24A and 24B are in the open position, as shown in FIGS. 2B and 3A-B, and when in the open position, such as shown in FIG. 4C.

Reference is made to FIGS. 5A-C. For some applications, electrosurgical instrument 20 is configured such that when first and second jaws 24A and 24B are in the closed position sandwiching the inserted leaflet portion and the electrical current is applied to electrically exposed cutting portion 36 by electrosurgical generator unit 22, pulling of first and second electrically insulated wire portions 34A and 34B in a proximal direction 66 into the one or more longitudinal channels 28 pulls electrically exposed cutting portion 36 along predefined path 42. Alternatively, for some applications, pushing of first and second electrically insulated wire portions 34A and 34B in a distal direction 68 from the one or more longitudinal channels 28 pushes electrically exposed cutting portion 36 along predefined path 42, in which case the illustrated sequence of locations of electrically exposed cutting portion 36 along predefined path 42 is from the location shown in FIG. 5C to the location shown in FIG. 5A.

Reference is still made to FIGS. 5A-C. For some applications, electrosurgical instrument 20 is configured such that when first and second jaws 24A and 24B are in the closed position sandwiching the inserted leaflet portion and the electrical current is applied to electrically exposed cutting portion 36 by electrosurgical generator unit 22, pulling of first and second electrically insulated wire portions 34A and 34B in proximal direction 66 into the one or more longitudinal channels 28 pulls electrically exposed cutting portion 36 along predefined path 42 such that electrically exposed cutting portion 36 first travels along predefined path 42 away from a junction 69 between (a) first and second jaws 24A and 24B and (b) delivery shaft 26, such as shown in the transition from FIG. 5A to FIG. 5B, and thereafter travels along predefined path 42 toward junction 69, such as shown in FIG. 5C.

Reference is made to FIGS. 2A-B, 3A-B, and 5A-C. For some applications, first and second grooves 50A and 50B are aligned with each other when first and second jaws 24A and 24B are in the closed position.

Reference is made to FIGS. 2A-B, 3A-B, 4A-C, and 5A-C. For some applications, as labeled in FIG. 4C, ends 70A of first groove 50A are at an end 72A of first leaflet-facing surface 40A that is supported at distal end portion 30 of delivery shaft 26, and ends 70B of second groove 50B are at an end 72B of second leaflet-facing surface 40B that is supported at distal end portion 30 of delivery shaft 26.

Reference is made to FIGS. 2A-B, 3A-B, 4A-C, and 5A-C. For some applications, no portion of electrically exposed cutting portion 36 is within first groove 50A and no portion of electrically exposed cutting portion 36 is within second groove 50B.

For some applications, electrically exposed cutting portion 36 has a non-circular cross-section perpendicular to a longitudinal axis thereof, such as to form a sharp edge that facilitates the cutting of the leaflet. For example, the non-circular cross-section may have a shape selected from the group consisting of: a U-shape, a V-shape, a triangular shape, a diamond shape, and a horseshoe shape.

For some applications, as shown in the figures, electrically exposed cutting portion 36 integrally defines its cutting surface. For other applications, electrically exposed cutting portion 36 comprises a discrete cutting surface that is fixed to (e.g., by soldering or crimping) the portion of wire 32 that defines electrically exposed cutting portion 36 (configuration not shown). For example, the discrete cutting surface may have the shape of a small cylinder.

For some applications, such as can be seen in FIGS. 2B, 3A, 4A, 4C, 6, and 8B, one of first and second jaws 24A and 24B is shaped so as to define a guidewire channel 74. By way of example, second jaw 24B is shown as defining guidewire channel 74. The guidewire channel may be used as described hereinbelow with reference to FIGS. 8A-G. The guidewire channel typically continues through all or a portion of delivery shaft 26. Typically, the guidewire channel has a diameter of between 0.035′ and 0.042′, in order to accommodate a standard guidewire having a diameter of 0.035′-0.038′.

For some applications, electrosurgical instrument 20 further comprises a channel having a distal opening near first and second jaws 24A and 24B for delivery of a non-electrically-conductive liquid. For example, the channel may be defined by deployment sheath 322, described hereinbelow, in which case the distal opening of the channel may be defined at a distal end of deployment sheath 322.

Reference is now made to FIG. 6, which is a schematic illustration of a portion of an electrosurgical instrument 120 with first and second jaws 124A and 124B thereof in an open position, in accordance with an application of the present invention. Except as described below, electrosurgical instrument 120 is identical to electrosurgical instrument 20, described hereinabove with reference to FIGS. 1-5C and hereinbelow with reference to FIGS. 8A-G, and may implement any of the features thereof, mutatis mutandis. Electrosurgical instrument 120 may also optionally implement any of the features of electrosurgical instrument 220, described hereinbelow with reference to FIG. 7, mutatis mutandis.

In this configuration, first and second jaws 124A and 124B are slidingly connected to each other. When slid together, the jaws grasp the leaflet portion in a manner similar to a vise.

For some applications, first and second jaws 124A and 124B are configured to slidingly move with respect to each other while respective planes 176A and 176B defined by first and second leaflet-facing surfaces 40A and 40B remain parallel to each other.

Reference is made to FIGS. 4A-C and 6. In some applications, at least one of first and second jaws 24A and 24B or 124A and 124B comprises one or more teeth 180 (e.g., exactly two teeth 180) that are configured to pierce the leaflet segment to be excised, when the first and the second jaws are in the closed position, so as to fixate the excised leaflet segment between the first and the second jaws. This fixation may help hold the leaflet segment in place during the cutting procedure and/or may ensure that the entire excised leaflet segment is removed from the vasculature with the jaws when they are withdrawn from the body, as described hereinbelow with reference to FIG. 8G. For example, one of the jaws may comprise one or more teeth 180 (such as shown), or each of the jaws may comprise one or more teeth 180 (configuration not shown).

The one or more teeth 180 are typically sharp.

Optionally, the one or more teeth 180 protrude from one or both of first and second leaflet-facing surfaces 40A and 40B, such as shown for first leaflet-facing surface 40A in FIGS. 1, 2A-B, 3B, 4A-C, 5A-C, 6, 7, 8B-F, and 10. For some application, two or more teeth 180 are located alongside one another at a same axial location along the jaw (or jaws), such as shown. For other applications, two or more teeth 180 are located at different axial locations along the jaw (or jaws) (configuration not shown).

Optionally, the opposite jaw is shaped so as to define one or more corresponding receptacles 182 for insertion of the one or more teeth 180, respectively, such as shown (labeled in FIGS. 1 and 4B).

Reference is now made to FIGS. 4D and 4E-F, which are schematic isometric and cross-sectional illustrations of first and second jaws 24A and 24B of another configuration of electrosurgical instrument 20, in accordance with an application of the present invention. The features of this configuration may optionally be implemented in any of the electrosurgical instruments described herein, mutatis mutandis.

In this configuration, at least one of first and second jaws 24A and 24B comprises a plurality of teeth 181 (e.g., 10 or more teeth, no more than 100 teeth, and/or 10-100 teeth) that are configured to grasp (and optionally pierce) the leaflet segment to be excised (such as by squeezing and/or by friction), when the first and the second jaws are in the closed position, so as to fixate the excised leaflet segment between the first and the second jaws. This fixation may help hold the leaflet segment in place during the cutting procedure and/or may ensure that the entire excised leaflet segment is removed from the vasculature with the jaws when they are withdrawn from the body, as described hereinbelow with reference to FIG. 8G. Alternatively or additionally, teeth 181 may cause partial blood isolation of the tissue within the jaws from outside the jaws.

For example, each of the jaws may comprise one or more teeth 181, which are optionally interlocking with opposite teeth (such as shown), or one of the jaws may comprise one or more teeth 180 (configuration not shown).

Typically, teeth 181 are disposed near a periphery of the jaw or jaws. (Although teeth 181 are shown disposed only along lateral portions of the jaws, teeth 181 may alternatively or additionally be disposed along the end of one or both of the jaws (configuration not shown).

Optionally, teeth 181 are arranged with a density of 1-2 teeth per millimeter.

Reference is now made to FIG. 7, which is a schematic illustration of a portion of a bipolar electrosurgical instrument 220 with first and second jaws 224A and 224B thereof in an open position, in accordance with an application of the present invention. Except as described below, electrosurgical instrument 220 is identical to electrosurgical instrument 20, described hereinabove with reference to FIGS. 1-5C and hereinbelow with reference to FIGS. 8A-G, and may implement any of the features thereof, mutatis mutandis. Electrosurgical instrument 220 may also optionally implement any of the features of electrosurgical instrument 120, described hereinbelow with reference to FIG. 6, mutatis mutandis.

In this configuration, one of first and second jaws 24A and 24B comprises a return electrode 284, which is configured to be coupled in electrical communication with electrosurgical generator unit 22 to provide bipolar electrical current between electrically exposed cutting portion 36 and return electrode 284. Return electrode 284 helps direct the current applied using electrosurgical generator unit 22 through tissue of the inserted leaflet portion.

Reference is now made to FIGS. 8A-G, which are schematic illustrations of a method of excising a segment 300 of a native or prosthetic heart valve leaflet 302 of a patient, in accordance with an application of the present invention. Although the method is illustrated using electrosurgical instrument 20, the method may alternatively be performed using electrosurgical instrument 120 or 220, described hereinabove with reference to FIGS. 6 and 7, respectively, mutatis mutandis. (Segment 300 is labeled in FIG. 8G.)

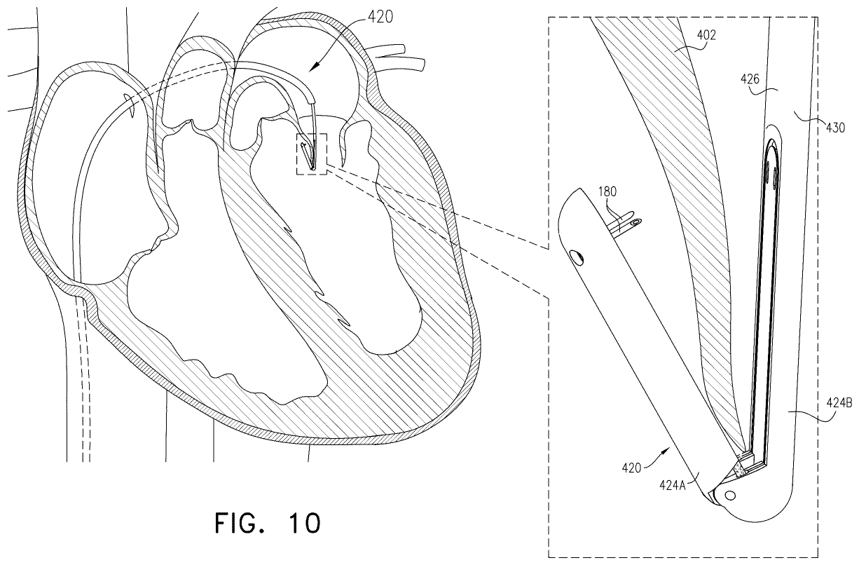

The method is typically performed before performing a transcatheter valve implantation, such as a Transcatheter Aortic Valve Implantation (TAVI), as shown in FIGS. 8A-G, or a Transcatheter Mitral Valve Replacement (TMVR) procedure for mitral valve implantation, as shown in FIG. 10, described hereinbelow, in order to assist with the implantation of an artificial valve. The method is typically performed with the aid of fluoroscopic X-ray imaging, similar to cardiac catheterization procedures. Although the method is illustrated as being performed on an aortic valve 304, the method may also be performed, mutatis mutandis, on another cardiac valve, such as a mitral valve before performance of TMVR, such as described hereinabove with reference to FIG. 10.

As shown in FIG. 8A, electrically exposed cutting portion 36 is electrically coupled to electrosurgical generator unit 22 via at least one of first and second electrically insulated wire portions 34A and 34B.

As also shown in FIG. 8A, delivery shaft 26 of electrosurgical instrument 20 is advanced through vasculature 306 of the patient to the native or prosthetic heart valve leaflet 302, typically while first and second jaws 24A and 24B are in the closed position. FIG. 8A also shows a left coronary artery 308 and a right coronary artery 310.

Typically, electrosurgical instrument 20 is advanced through vasculature 306 over a guidewire 312 that has been inserted into a left ventricle 314, e.g., via the femoral artery. Guidewire 312 is typically not an element of electrosurgical instrument 20. For example, first an introducer sheath (separate from deployment sheath 322 described below) may be inserted into the femoral artery. The aortic valve may be crossed with a straight wire over a catheter (e.g., an AL1 catheter) and an exchange wire may be introduced to left ventricle 314. Then a pigtail catheter may be introduced, and the guidewire 312 is inserted to the apex of left ventricle 314.

Electrosurgical instrument 20 is then advanced over guidewire 312 to a location above the aortic root. Guidewire 312 typically passes through guidewire channel 74, which, as described hereinabove, is defined by one of first and second jaws 24A and 24B.

For some applications, electrosurgical instrument 20 comprises a deployment sheath 322 and/or a nosecone 324, such as shown in FIG. 8A-G. Typically, a distal portion of deployment sheath 322 is configured to be steerable for guidance of deployment sheath 322 to the target location, typically with the aid of fluoroscopic X-ray imaging, and/or echocardiography, similar to cardiac catheterization procedures, as is known in the art.

For other applications, electrosurgical instrument 20 does not comprise deployment sheath 322 and/or does not comprise nosecone 324.

As shown in FIG. 8B, optionally, after nosecone 324 of electrosurgical instrument 20 has been advanced into left ventricle 314, deployment sheath 322 is proximally retracted.

As shown in FIG. 8C, electrosurgical instrument 20 is advanced out of a distal end of deployment sheath 322 toward leaflet 302 of the aortic valve. Although the aortic valve is shown in the figures as a native aortic valve, the aortic valve may also be a prosthetic valve of a previously implanted prosthetic aortic valve, e.g., implanted in a TAVI procedure.

As shown in FIG. 8D, after confirmation of proper positioning, first and second jaws 24A and 24B are spaced apart to assume the open position. For some applications, electrosurgical instrument 20 comprises a spring that is configured to separate first and second jaws 24A and 24B from each other; alternatively, first and second jaws 24A and 24B are opened using other techniques, such as using one or more control wires.

Electrosurgical instrument 20 is oriented towards the leaflet 302 that needs laceration. As also shown in FIG. 8D, electrosurgical instrument 20 is advanced into contact with leaflet 302, so as to insert, between first and second jaws 24A and 24B, a portion of leaflet 302 including leaflet segment 300 to be excised, such that first and second jaws 24A and 24B are disposed on opposite sides of the leaflet. The portion of leaflet 302 also typically includes a free edge of leaflet 302. Typically, first jaw 24A is disposed on an aortic side of leaflet 302 and second jaw 24B is disposed on a ventricular side of leaflet 302 (as mentioned above, first jaw 24A may be moveable with distal end portion 30 of delivery shaft 26, while second jaw 24B may be fixed with respect to distal end portion 30).

As shown in FIG. 8E, first and second jaws 24A and 24B are transitioned to the closed position, in which first and second jaws 24A and 24B sandwich the inserted leaflet portion.

As shown in FIG. 8F, electrosurgical generator unit 22 is activated to apply an electrical current to electrically exposed cutting portion 36, and leaflet segment 300 is excised from the inserted leaflet portion by moving electrically exposed cutting portion 36 along predefined path 42 (for example, as described hereinabove with reference to FIGS. 5A-C), such that excised leaflet segment 300 remains entrapped between first and second jaws 24A and 24B, as shown in the cross-sectional blow-up in FIG. 8G. The power and electrical wave-form applied by electrosurgical generator unit 22 are selected to optimize the excision.

For some of these applications, electrically exposed cutting portion 36 is moved along predefined path 42 by pulling electrically exposed cutting portion 36 along predefined path 42, by pulling first and second electrically insulated wire portions 34A and 34B in proximal direction 66 into the one or more longitudinal channels 28.

For some applications, a non-electrically-conductive liquid is delivered via a channel having a distal opening near first and second jaws 24A and 24B, so as to dilute blood in a vicinity of the first and the second jaws. This dilution of the blood reduces the electrical conductivity of the blood in the vicinity of the jaws, which helps focus the electrical current flow through the leaflet tissue and reduces the bypass of the current through the blood. For example, the non-electrically-conductive liquid may comprise glucose solution.

As shown in FIG. 8G, electrosurgical instrument 20 is removed from the patient while excised leaflet segment 300 remains entrapped between first and second jaws 24A and 24B, typically after proximally retracting first and second jaws 24A and 24B into deployment sheath 322. Deployment sheath 322, including first and second jaws 24A and 24B, is withdrawn from the patient's body.

Typically, an artificial aortic valve is subsequently implanted, such as in a TAVI procedure.

Reference is made to FIGS. 9A-B, which are schematic illustrations of leaflet 302 after it has been excised using electrosurgical instrument 20 as described with reference to FIGS. 8A-G, in accordance with an application of the present invention. As can be seen, the ostium of right coronary artery 310 is not obstructed when leaflet 302 is pushed toward the aorta, such as during implantation of a prosthetic valve, e.g., during a TAVI procedure.

For some applications, a return electrode is applied to the patient's body outside the heart, such as on an external surface of the skin, and electrosurgical generator unit 22 is activated to provide monopolar electrical current between electrically exposed cutting portion 36 and the remote return electrode. The return electrode may or may not be an element of electrosurgical instrument 20. For other applications, the electrosurgical instrument comprises return electrode 284, described hereinabove regarding electrosurgical instrument 220 with reference to FIG. 7, and electrosurgical generator unit 22 is activated to provide bipolar electrical current between electrically exposed cutting portion 36 and return electrode 284.

For some applications, excised leaflet segment 300 has a surface area of 3-60 mm2, e.g., 3-30 mm2, such as 3-10 mm2. To this end, for these applications, electrosurgical instrument 20 is configured such that the movement of electrically exposed cutting portion 36 along predefined path 42 excises leaflet segment 300 having the surface area of 3-60 mm2, e.g., 3-30 mm2, such as 3-10 mm2.

Reference is made to FIGS. 9A-B, which are schematic illustration of leaflet 302 after the piece of material has been removed using electrosurgical instrument 20 as described with reference to FIGS. 8A-G, in accordance with an application of the present invention. As can be seen, the ostium of right coronary artery 310 is not obstructed when leaflet 302 is pushed toward the aorta, such as during implantation of a prosthetic valve, e.g., during a TAVI procedure.

Reference is now made to FIG. 10, which is a schematic illustration of a portion of an electrosurgical instrument 420 and of a portion of a method of excising a segment 400 of a native mitral valve leaflet 402 of a patient using electrosurgical instrument 420, in accordance with an application of the present invention. Other than as described below, electrosurgical instrument 420 is similar to electrosurgical instrument 20, described hereinabove. Optionally, electrosurgical instrument 420 may implement features of electrosurgical instrument 120 and/or 220, described hereinabove with reference to FIGS. 6 and 7, respectively, mutatis mutandis. The method is typically performed before performing Transcatheter Mitral Valve Replacement (TMVR), in order to assist with the implantation of an artificial valve by preventing left ventricle outward tract obstruction that may be caused by the native mitral anterior leaflet.

For applications in which a transseptal approach is used, such as shown in FIG. 10, first and second jaws 424A and 424B of electrosurgical instrument 420, when in the closed position, face proximally toward a distal end portion 430 of a delivery shaft 426 of electrosurgical instrument 420, rather than distally away from distal end portion 30 of delivery shaft 26, as in electrosurgical instruments 20, 120, and 220. The jaws are advanced into the ventricle and then withdrawn proximally and positioned on opposite atrial and ventricular sides of mitral valve leaflet 402. Typically, first jaw 424A is disposed on a ventricular side of leaflet 402 and second jaw 424B is disposed on an atrial side of leaflet 402 (as mentioned above regarding first and second jaws 24A and 24B of electrosurgical instrument 20, first jaw 424A may be moveable with distal end portion 430 of delivery shaft 426 of electrosurgical instrument 420, while second jaw 424B may be fixed with respect to distal end portion 430).

The remainder of the method is performed as described hereinbelow with reference to FIGS. 8A-G, mutatis mutandis. Typically, mitral valve leaflet 402 is the anterior leaflet, such as a middle segment (A2) thereof, which is lacerated using electrosurgical instrument 420 in order to prevent iatrogenic left ventricular outflow tract (LVOT) obstruction immediately before TMVR.

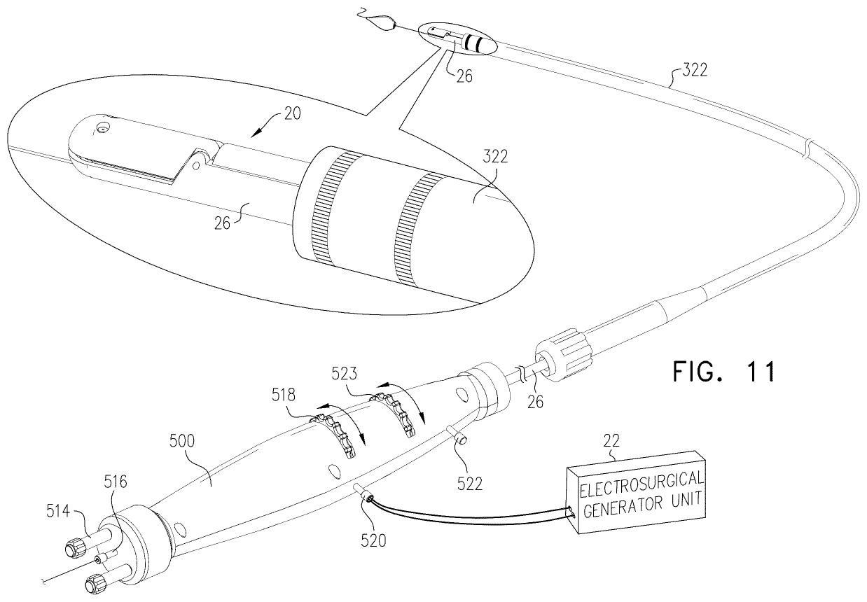

Reference is now made to FIG. 11, which is a schematic illustration of an operating handle 500 of electrosurgical instrument 20, in accordance with an application of the present invention. Operating handle 500 is positioned at a proximal end of delivery shaft 26 of electrosurgical instrument 20, and at a proximal end of deployment sheath 322 if provided. Although operating handle 500 is shown in FIG. 11 as positioned at the proximal end of delivery shaft 26 of electrosurgical instrument 20, the operating handle may also be positioned at the proximal end of the delivery shaft connected to electrosurgical instruments 120, 220, and 420, mutatis mutandis.

Operating handle 500 typically has several functions, such as delivery, deployment, activation, retrieval, steering (e.g., in two dimension), and/or opening and closing jaws 24A and 24B. To this end, operating handle 500 typically comprises one or more of the following elements:
a knob/port 514 through which first and second electrically insulated wire portions 34A and 34B is moved (e.g., pulled), so as to move (e.g., pull) the insulated wire portions within the one or more longitudinal channels 28;
a knob/port 516 for guidewire 312;
a knob 518 for opening and closing jaws 24A and 24B; for example, rotation of knob 518 in one direction pull on a control wire coupled to one or both of the jaws (such as the upper jaw), and rotation of the knob in the opposite direction may push on the control wire; the pushing and pulling of the control wire may close and open the jaws; optionally knob 518 rotates a screw inside operating handle 500 so that the tension of the control wire remains constant when the knob is stationary, thereby locking the position of the jaws;
a port 520 for electrical coupling to electrosurgical generator unit 22;
a flushing port 522; and/or
a knob 523 for steering a distal portion of deployment sheath 322 (such as the distal-most 15 cm of deployment sheath 322). For example, by turning the handle, e.g., sideways, the operator can control the distal portion of deployment sheath 322 from a straight direction to a 90-degree angle (similar to a trunk of an elephant). For example, this may enable steering the deployment sheath while it is advanced through the aortic arch and pointing it directly onto the selected cusp of the aortic valve. Optionally, two knobs 523 are provided, which respectively control steering about two perpendicular axes (configuration not shown).

Alternatively, such as in configurations in which knob 523 is not provided, steering may be performed by turning of handle 500, which may enable steering of the distal portion of deployment sheath 322, as described above regarding knob 523.

Typically, delivery shaft 26 and deployment sheath 322 can be advanced separately, using operating handle 500, once deployment sheath 322 has reached its final position, typically a few centimeters from the valve (similar to an endoscope and a biopsy tool which is advanced through its working channel).

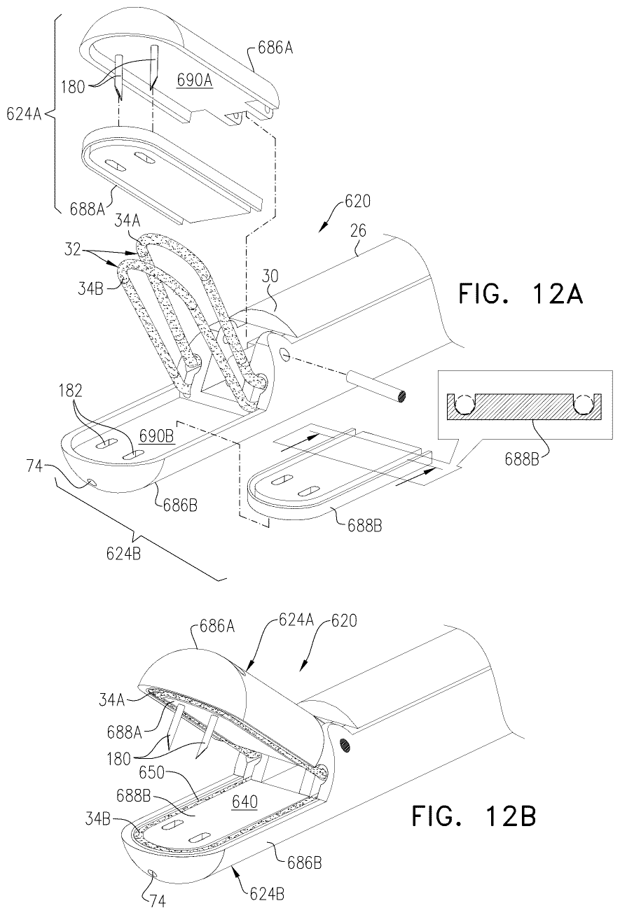

Reference is now made to FIGS. 12A-B, which are schematic illustrations of a portion of an electrosurgical instrument 620 for use with electrosurgical generator unit 22, for excising a segment of a native or prosthetic heart valve leaflet, in accordance with an application of the present invention. FIG. 12A is an exploded view, and FIG. 12B shows with electrosurgical instrument 620 with first and second jaws 624A and 624B thereof in an open position. Except as described below, electrosurgical instrument 620 is identical to electrosurgical instrument 20, described hereinabove with reference to FIGS. 1-5C and 8A-G, and may implement any of the features thereof, mutatis mutandis; like parts refer to like elements. Electrosurgical instrument 620 may also optionally implement any of the features of the other electrosurgical instruments described herein. Similarly, any of the electrosurgical instruments described herein may implement the features of electrosurgical instrument 620.

As mentioned above regarding electrosurgical instrument 20 with reference to FIGS. 4A-C, first and second jaws 24A and 24B of electrosurgical instrument 20 typically comprise an electrically insulating material, such that the current applied using electrosurgical generator unit 22 is directed through tissue of the inserted leaflet portion, rather than through material of the jaws.

By contrast, first and second jaws 624A and 624B of electrosurgical instrument 620 typically comprise:
respective first and second outer shells 686A and 686B, which comprise an electrically-conductive metal; and
respective first and second inserts 688A and 688B, which comprise an electrically insulating material (for example, the electrically insulating material may comprise a polymer, such as polyimide or polyether ether ketone (PEEK); or a ceramic material).

First and second outer shells 686A and 686B are shaped so as to define respective first and second insert receptacles 690A and 690B, which are shaped so as to receive first and second inserts 688A and 688B, respectively, and first and second inserts 688A and 688B are disposed within first and second insert receptacles 690A and 690B, respectively. First and second outer shells 686A and 686B and first and second inserts 688A and 688B thus together define first and second jaws 624A and 624B, respectively.

As labeled in FIG. 12B, first and second inserts 688A and 688B are shaped so as to define at least respective portions of respective first and second leaflet-facing surfaces 640 (typically respective majorities of the first and second leaflet-facing surfaces; respective peripheral portions of the leaflet-facing surfaces may be defined by peripheral portions of outer shells 686A and 686B). (Only the second leaflet-facing surface 640 can be seen in FIGS. 12A-B.)

For some applications, the at least respective portions of first and second leaflet-facing surfaces 640 defined by first and second inserts 688A and 688B, respectively, are shaped so as to define respective first and second grooves 650 (i.e., elongate indentations), which together define predefined path 42 when first and second jaws 624A and 624B are in the closed position. (Only the second groove 650 can be seen in FIGS. 12A-B.)

The metal of first and second outer shells 686A and 686B provides strength to the jaws, allowing the jaws to apply high force during closing of the jaws. The electrical insulation of first and second inserts 688A and 688B allows wire 32 or the elongate assembly to slide through groove 650 without electrically shorting to the metal outer shells.

For some applications, first and second inserts 688A and 688B are fixed within first and second receptacles 690A and 690B, typically during manufacture. For other applications, first and second inserts 688A and 688B are removably insertable into first and second receptacles 690A and 690B by a healthcare worker before or during a procedure, and are removable to allow re-use of some or all of the other components of electrosurgical instrument 620 with a separate set of inserts 688A and 688B for each procedure.

For some applications, only one of first and second jaws 624A and 624B comprises an outer shell 686 and an insert 688, such as described above. The other jaw typically comprises an electrically insulating material. For example, this configuration may allow for the jaw that comprises the metal outer shell 686 to apply more force than the other jaw that comprises a non-metal electrically insulating material, such as a polymer. For some of these applications, the at least a portion of the leaflet-facing surface 640 defined by the insert 688 is shaped so as to define a groove 650 (i.e., elongate indentations), which together with the groove 50 of the other leaflet-facing surface 40, define predefined path 42 when first and second jaws 624A and 624B are in the closed position.

Reference is now made to FIGS. 13A-B, which are schematic illustrations of respective configurations of a portion of an electrosurgical instrument 720 for use with electrosurgical generator unit 22, for excising a segment of a native or prosthetic heart valve leaflet, in accordance with respective applications of the present invention. FIGS. 13A-B show electrosurgical instrument 720 with first and second jaws 24A and 24B thereof in a closed position. For clarity of illustration, second jaw 24B is shown as transparent in FIGS. 13A-B. Except as described below, electrosurgical instrument 720 is identical to electrosurgical instrument 20, described hereinabove with reference to FIGS. 1-5C and 8A-G, and may implement any of the features thereof, mutatis mutandis; like parts refer to like elements. Electrosurgical instrument 720 may also optionally implement any of the features of the other electrosurgical instruments described herein. Similarly, any of the electrosurgical instruments described herein may implement the features of electrosurgical instrument 720.

As mentioned above regarding electrosurgical instrument 20 with reference to FIGS. 2A-B, 3A-B, 4A-C, and 5A-C, for some applications first electrically insulated wire portion 34A, electrically exposed cutting portion 36, and second electrically insulated wire portion 34B comprise respective three respective pieces fixed together (by an electrical and mechanical bonding/connection), which collectively define an elongate assembly. Similarly, electrosurgical instrument 720 comprises first and second electrically insulated wire portions 734A and 734B and, longitudinally therebetween, an electrically exposed cutting portion 736 that is coupled to first and second electrically insulated wire portions 734A and 734B, for example by soldering, so as to collectively define an elongate assembly 732.

For some applications, electrically exposed cutting portion 736 is shaped so as to define a sharp cutting surface 737 (electrically exposed cutting portion 736 may thus be considered a blade). Sharp cutting surface 737 faces in the direction of movement of electrically exposed cutting portion 36 along predefined path 42. FIGS. 13A-B shows a configuration in which electrosurgical instrument 720 is configured such that pulling of first and second electrically insulated wire portions 34A and 34B in proximal direction 66 into the one or more longitudinal channels 28 pulls electrically exposed cutting portion 36 along predefined path 42, such as shown and described hereinabove with reference to FIGS. 5A-C. Alternatively, sharp cutting surface 737 may face in the opposite direction, such as in configurations in which electrosurgical instrument 720 is configured such that pushing of first and second electrically insulated wire portions 34A and 34B in distal direction 68 from the one or more longitudinal channels 28 pushes electrically exposed cutting portion 36 along predefined path 42, such as described hereinabove with reference to FIGS. 5A-C (configuration not shown).

Electrically exposed cutting portion 736, including sharp cutting surface 737, is configured to provide effective and smooth cutting of heart valve leaflet, particularly if the heart valve leaflet is hardened by calcification.

For example, sharp cutting surface 737 may be straight, such as shown in FIG. 13A (labeled 737A), or another geometry, such as shown in FIG. 13B (labeled 737B), e.g., V-shaped as shown, or curved (e.g., horseshoe-shaped, arced, or crescent-shaped).

As described hereinabove with reference to FIG. 3A, for some applications, respective widths W_{A3} and W_{B3} of elongate assembly 732 at at least first and second longitudinal locations 56A and 56B along elongate assembly 732 are greater than the respective widths W_{A1} and W_{B1} of first and second longitudinal openings 54A and 54B. For some of these applications, first and second longitudinal locations 56A and 56B along elongate assembly 732 are defined by respective ends of electrically exposed cutting portion 736 (configuration not shown).

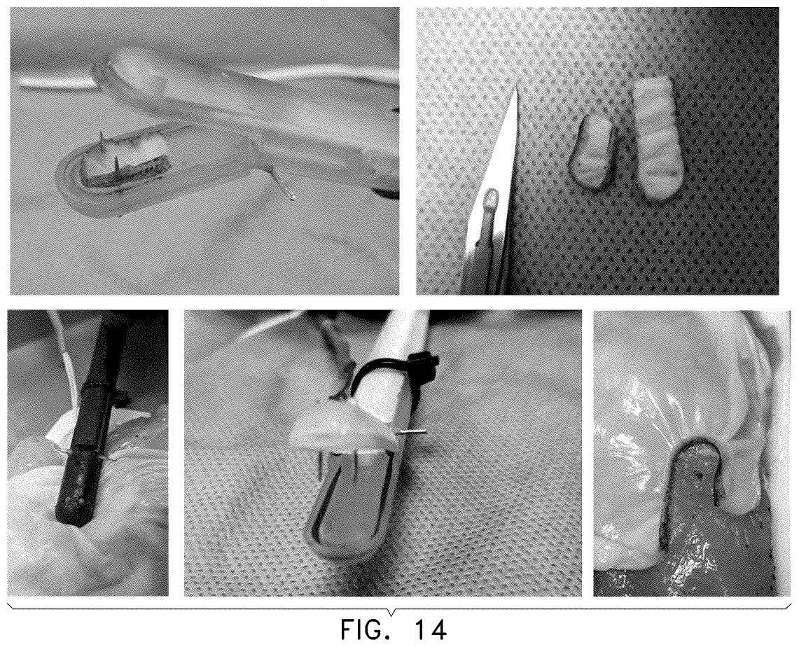

Reference is now made to FIG. 14, which includes several photographs of a prototype and experiments conducted by the inventors, in accordance with an application of the present invention. The inventors built a prototype similar to the electrosurgical instrument described herein and tested it in real life scenarios. The prototype was constructed from non-conductive solid polymer in 2:1 scale of the final electrosurgical instrument. The inventors integrated a 0.014″ guidewire (used in cardiology Cath-lab procedures) as the cutting wire after removing a few-millimeter section of its outer layer to expose the flexible internal metal wire. The wire was fitted into the U shape groove in both jaws so the it would follow that path when pulled. The two ends of the wire (top and bottom) were connected together to an electrosurgical generator unit which was set a 55 watts power level. Two metal pins (shaped like snake teeth) penetrated the tissue sample when the jaws were closed in order to secure the tissue sample after its excision.

Once the jaws were securely attached to the tissue, electrical current was applied to the cutting wire which was then gently pulled by hand, along its track, until the tissue specimen was fully excised.

Several experiments were conducted:
1. The jaws were attached to a sample of bovine pericardial tissue which was used to simulate the cusps of the prosthetic aortic valve.
2. The jaws were attached to the native aortic cusp of a porcine heart valve (ex vivo).
3. The jaws were attached to a sample of bovine pericardial tissue and the pericardial tissue was submerged in saline solution to simulate the aortic cusp surrounded by flowing blood.

In all three experiments, the tissue was cut smoothly and without causing damage to the surrounding tissue due to overheating. The cut out tissue segment was secured within the jaws as can be seen in the photographs of FIG. 14. Submersion of the pericardial tissue in saline solution did not adversely affect the electrosurgical cutting.

In an embodiment, techniques and apparatus described herein are combined with techniques and apparatus described in U.S. Provisional Application 63/051,479, filed Jul. 14, 2020, and/or International Application PCT/IL2021/050844, filed Jul. 11, 2021 (published as PCT Publication WO 2022/013857 to Feld et al.), which are assigned to the assignee of the present application and incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An electrosurgical instrument for use with an electrosurgical generator unit, for excising a segment of a native or prosthetic heart valve leaflet, the electrosurgical instrument comprising:
a delivery shaft, which is shaped so as to define one or more longitudinal channels therethrough;
first and second jaws, which (a) are supported at a distal end portion of the delivery shaft, (b) are shaped so as to define respective first and second leaflet-facing surfaces, and (c) together are configured to assume (i) an open position, in which the first and the second jaws are spaced apart to allow insertion, between the first and the second jaws, of a portion of the leaflet including the leaflet segment to be excised, and (ii) a closed position, in which the first and the second jaws sandwich the inserted leaflet portion; and
first and second electrically insulated wire portions and, longitudinally therebetween, an electrically exposed cutting portion, wherein at least one of the first and the second electrically insulated wire portions passes through the one or more longitudinal channels of the delivery shaft and is configured to couple the electrically exposed cutting portion in electrical communication with the electrosurgical generator unit,
wherein the electrosurgical instrument is configured such that when the first and the second jaws are in the closed position sandwiching the inserted leaflet portion and an electrical current is applied to the electrically exposed cutting portion by the electrosurgical generator unit, movement of the electrically exposed cutting portion along a predefined path excises the leaflet segment from the inserted leaflet portion, such that the excised leaflet segment remains entrapped between the first and the second jaws.

2. The electrosurgical instrument according to claim 1, wherein the electrosurgical instrument comprises a wire that comprises the first and the second electrically insulated wire portions and, longitudinally therebetween, the electrically exposed cutting portion.

3. The electrosurgical instrument according to claim 1, wherein the native or prosthetic valve is selected from the group consisting of a native or prosthetic aortic valve, and a native mitral valve, and the electrosurgical instrument is configured to excise the segment of the selected native or prosthetic valve.

4-12. (canceled)

13. The electrosurgical instrument according to claim 1, wherein one or both of the first and the second leaflet-facing surfaces are flat.

14-15. (canceled)

16. The electrosurgical instrument according to claim 1, wherein at least one of the first and the second jaws comprises a return electrode, which is configured to be coupled in electrical communication with the electrosurgical generator unit to provide bipolar electrical current between the electrically exposed cutting portion and the return electrode.

17-20. (canceled)

21. The electrosurgical instrument according to claim 1, wherein one of the first and the second jaws is shaped so as to define a guidewire channel.

22. The electrosurgical instrument according to claim 1, further comprising a channel having a distal opening near the first and the second jaws for delivery of a non-electrically-conductive liquid.

23. The electrosurgical instrument according to claim 1,
wherein the first jaw comprises (a) a first outer shell, which comprises an electrically-conductive metal; and (b) a first insert, which comprises an electrically insulating material,
wherein the first outer shell is shaped so as to define a first insert receptacle, which is shaped so as to receive the first insert, and the first insert is disposed within the first insert receptacle,
wherein the first insert is shaped so as to define at least a portion of the first leaflet-facing surface,
wherein the second jaw comprises (a) a second outer shell, which comprises an electrically-conductive metal; and (b) a second insert, which comprises an electrically insulating material,
wherein the second outer shell is shaped so as to define a second insert receptacle, which is shaped so as to receive the second insert, and the second insert is disposed within the second insert receptacle, and
wherein the second insert is shaped so as to define at least a portion of the second leaflet-facing surface.

24. (canceled)

25. The electrosurgical instrument according to claim 23,
wherein the at least respective portions of the first and the second leaflet-facing surfaces defined by the first and the second inserts, respectively, are shaped so as to define respective first and second grooves, which together define the predefined path when the first and the second jaws are in the closed position,
wherein, at least when the first and the second jaws are in the closed position, the first and the second electrically insulated wire portions are slidably disposed partially within the first and the second grooves, respectively, with the electrically exposed cutting portion extending between respective planes defined by the first and the second grooves, and
wherein the electrosurgical instrument is configured such that when the first and the second jaws are in the closed position sandwiching the inserted leaflet portion and the electrical current is applied to the electrically exposed cutting portion by the electrosurgical generator unit, motion of the first and the second electrically insulated wire portions in the one or more longitudinal channels causes the movement of the electrically exposed cutting portion along the predefined path.

26-27. (canceled)

28. The electrosurgical instrument according to claim 1, wherein, at least when the first and the second jaws are in the closed position, the electrically exposed cutting portion includes a curved segment therealong.

29-34. (canceled)

35. The electrosurgical instrument according to claim 1,
wherein the first and the second leaflet-facing surfaces are shaped so as to define respective first and second grooves, which together define the predefined path when the first and the second jaws are in the closed position,
wherein, at least when the first and the second jaws are in the closed position, the first and the second electrically insulated wire portions are slidably disposed partially within the first and the second grooves, respectively, with the electrically exposed cutting portion extending between respective planes defined by the first and the second grooves, and
wherein the electrosurgical instrument is configured such that when the first and the second jaws are in the closed position sandwiching the inserted leaflet portion and the electrical current is applied to the electrically exposed cutting portion by the electrosurgical generator unit, motion of the first and the second electrically insulated wire portions in the one or more longitudinal channels causes the movement of the electrically exposed cutting portion along the predefined path.

36. The electrosurgical instrument according to claim 35,
wherein the electrosurgical instrument comprises a wire that comprises the first and the second electrically insulated wire portions and, longitudinally therebetween, the electrically exposed cutting portion,
wherein the first and the second grooves are shaped so as to define respective first and second longitudinal openings, which have respective widths that are narrower than greatest respective widths of the first and the second grooves,
wherein respective widths of the wire at at least first and second longitudinal locations along the wire are greater than the respective widths of the first and the second longitudinal openings, and
wherein a narrowest width of the electrically exposed cutting portion is less than the respective widths of the first and the second longitudinal openings.

37. The electrosurgical instrument according to claim 35,
wherein the electrosurgical instrument comprises a wire that comprises the first and the second electrically insulated wire portions and, longitudinally therebetween, the electrically exposed cutting portion,
wherein the first and the second grooves are shaped so as to define respective first and second longitudinal openings, which have respective widths that are narrower than greatest respective widths of the first and the second grooves,
wherein the wire comprises first and second sliding beads fixed at first and second longitudinal locations along the wire,
wherein the first and the second sliding beads are configured to slide through the first and the second grooves and have respective widths that are greater than the respective widths of the first and the second longitudinal openings, and
wherein a narrowest width of the electrically exposed cutting portion is less than the respective widths of the first and the second sliding beads.

38-42. (canceled)

43. The electrosurgical instrument according to claim 35, wherein the first and the second grooves are not entirely straight therealong.

44. The electrosurgical instrument according to claim 43, wherein the first and the second grooves are U-shaped therealong.

45. The electrosurgical instrument according to claim 43, wherein the first and the second grooves are shaped so as to define one or more corresponding curved sections, respectively, which fall in the planes defined by the first and the second grooves, respectively.

46. (canceled)

47. The electrosurgical instrument according to claim 43, wherein the first and the second grooves are shaped so as to define respective corresponding straight sections.

48. The electrosurgical instrument according to claim 35, wherein the first and the second electrically insulated wire portions are slidably disposed partially within the first and the second grooves, respectively, both when the first and the second jaws are in the open position and are in the closed position.

49-50. (canceled)

51. The electrosurgical instrument according to claim 35, wherein the electrosurgical instrument is configured such that when the first and the second jaws are in the closed position sandwiching the inserted leaflet portion and the electrical current is applied to the electrically exposed cutting portion by the electrosurgical generator unit, pulling of the first and the second electrically insulated wire portions in a proximal direction into the one or more longitudinal channels pulls the electrically exposed cutting portion along the predefined path.

52. The electrosurgical instrument according to claim 51, wherein the electrosurgical instrument is configured such that when the first and the second jaws are in the closed position sandwiching the inserted leaflet portion and the electrical current is applied to the electrically exposed cutting portion by the electrosurgical generator unit, pulling of the first and the second electrically insulated wire portions in the proximal direction into the one or more longitudinal channels pulls the electrically exposed cutting portion along the predefined path such that the electrically exposed cutting portion first travels along the predefined path away from a junction between (a) the first and the second jaws and (b) the delivery shaft, and thereafter travels along the predefined path toward the junction.

53. The electrosurgical instrument according to claim 52, wherein the electrosurgical instrument is configured such that the predefined path is U-shaped.

54-57. (canceled)

58. The electrosurgical instrument according to claim 35,
wherein ends of the first groove are at an end of the first leaflet-facing surface that is supported at the distal end portion of the delivery shaft, and
wherein ends of the second groove are at an end of the second leaflet-facing surface that is supported at the distal end portion of the delivery shaft.

59. (canceled)

60. A method of excising a segment of a native or prosthetic heart valve leaflet of a patient, the method comprising:
electrically coupling, to an electrosurgical generator unit, an electrically exposed cutting portion of an electrosurgical instrument via at least one of first and second electrically insulated wire portions that longitudinal surround the electrically exposed cutting portion;
advancing a delivery shaft of the electrosurgical instrument through vasculature of the patient to the native or prosthetic heart valve leaflet, wherein the delivery shaft is shaped so as to define one or more longitudinal channels therethrough, wherein the at least one of the first and the second electrically insulated wire portions passes through the one or more longitudinal channels, and wherein the delivery shaft supports, at a distal end portion thereof, first and second jaws that are shaped so as to define respective first and second leaflet-facing surfaces;
while the first and second jaws are in an open position, in which the first and the second jaws are spaced apart, advancing the first and the second jaws so as to insert, between the first and the second jaws, a portion of the leaflet including the leaflet segment to be excised, such that the first and the second jaws are disposed on opposite sides of the leaflet;
transitioning the first and the second jaws to a closed position, in which the first and the second jaws sandwich the inserted leaflet portion;
thereafter, activating the electrosurgical generator unit to apply an electrical current to the electrically exposed cutting portion;
excising the leaflet segment from the inserted leaflet portion by moving the electrically exposed cutting portion along a predefined path, such that the excised leaflet segment remains entrapped between the first and the second jaws; and
thereafter, removing the electrosurgical instrument from the patient while the excised leaflet segment remains entrapped between the first and the second jaws.
